(19) United States
(12) Patent Application Publication
(10) Pub. No.: US 2024/0257963 A1
(43) Pub. Date: Aug. 1, 2024

(54) METHOD AND SYSTEM FOR PROVIDING SETTING PARAMETER VALUES TO A MEDICAL APPARATUS

(71) Applicant: GAMBRO LUNDIA AB, Lund (SE)

(72) Inventors: Nhi Nguyen, Blaine, MN (US); Derek Neiman, Bethel, MN (US); Hui Hoang, Maple Grove, MN (US); Taylor Piechowski, Champlin, MN (US)

(21) Appl. No.: 18/289,643

(22) PCT Filed: May 9, 2022
(86) PCT No.: PCT/EP2022/062482

§ 371 Date: Nov. 6, 2023

(30) Foreign Application Priority Data
May 11, 2021 (IT) 102021000012110

(51) Int. Cl. G16H 40/67; G16H 20/40

(52) U.S. Cl. CPC G16H 40/67; G16H 20/40

(57) ABSTRACT

A method and a related system for providing setting parameter values to a medical apparatus to perform a medical treatment, the method comprising a remote procedure comprising the steps of providing a remote device comprising a user interface, entering setting parameter values into the remote device by the user interface, wherein the setting parameter values define a setting of the medical apparatus and are related to an identified patient and are part of a patient prescription for said identified patient. The remote procedure further comprises generating a setting signal encoding said setting parameter values. The method further comprises an acquisition procedure including providing the medical apparatus with a reader, reading the setting signal by relatively approaching the reader of the medical apparatus to the remote device, and acquiring said setting parameter values through the reading step. The medical apparatus is set based on the values of said setting parameter values.

### FIELD OF THE INVENTION

The present invention relates to a method and a related system for wirelessly providing setting parameter values to a medical apparatus, e.g., to an extracorporeal blood treatment apparatus. The setting parameter values are used by the medical device, such as the extracorporeal blood treatment apparatus, to set and thereafter perform a medical treatment on a patient.

### BACKGROUND

Medical apparatuses, and especially extracorporeal blood treatment apparatuses, are set by operators, such as nurses, practitioners, or clinicians, in order to perform the proper medical treatment on the patient. In particular in dialysis field, a number of setting parameter values have to be first decided, defining the prescription, and then provided to the medical apparatus so that the apparatus operates according the prescription. In dialysis field, the setting parameter values may comprise the dialysis treatment type which is going to be performed, such as an ultrafiltration treatment (UF), a haemodialysis treatment (HD), a haemofiltration treatment (HF), haemodiafiltration treatment (HDF). Then for each of these types of dialysis treatments, further related setting parameter values may have to be entered such as fluid flow rates, treatment time and ultrafiltration rate. In other terms, the operator has to responsibility to reach each patient and manually entering the related setting parameter values in the medical apparatus. In this case the operator has to enter the setting parameter values through a user interface physically at the medical apparatus.

Alternatively, the step of entering the setting parameter values may also be performed by using a remote device, such as a remote computer, connected to the medical apparatus by a wired connection or alocal communication net, i.e. an intranet. In this case, the operator may enter the setting parameter values through a user interface of the remote device.

Anyhow, in both cases, the method and the related system to provide the setting parameter values to the medical apparatus is very rigid and leads to disadvantages which negatively affect the setting procedure. In particular, the user interface of the medical apparatus is updateable, but as part of a medical apparatus, implies consequent severe re-certifications. Furthermore, when setting the device, the user has to be physically located at the medical apparatus and, thus close to each patient, to enter the setting parameter values in the medical apparatus.

In the alternative wherein the user interface is remotely connected, the operator may set the medical apparatus remotely, but this may also negatively affect safety of the patient as the operator is not physically close to the patient.

### OBJECTIVE OF THE INVENTION

The object of this invention is therefore to at least partially solve one or more of the drawbacks and/or limitations of the previous solutions.

A first object is to provide a flexible method and related system to set the medical apparatus.

A further object is to provide a setting method and related setting system easy to be updated and modified, keeping at the same time reliability and safety of the medical apparatus.

An auxiliary aim is to provide a setting method and related setting system to allow the operator to set the medical apparatus close to the patient as fast as possible, in order to avoid long time exposure of the operator to a patient potentially affected by a transmissible disease.

An auxiliary aim is to provide a setting method and related setting system to allow the operator to enter the setting parameter values both remotely and locally with respect to the medical apparatus, so that the operator may type most of the parameters remotely in comfort and make changes to the setting parameter values when close to the patient.

A further auxiliary aim is to provide a setting method and related setting system to make the setting procedures easier, faster, and safer for the operator, keeping at the same time reliability and safety of the medical apparatus.

A further auxiliary aim is to provide a setting method and related setting system to make the setting procedures more reliable, by avoiding typing errors of the setting parameter values.

A further aim is to provide a setting method and related setting system for chronic or acute hemodialysis (HD) therapy.

These objects, aim and more, which will appear more from the following description, are substantially achieved by a system and a setting method in accordance with one or more of the following claims and/or aspects.

### SUMMARY SECTION

A first aspect refers to a method (1000) for providing setting parameter values to a medical apparatus to perform a medical treatment, the method comprising a remote procedure comprising the steps of:
providing a remote device (60) comprising a user interface (61);
entering setting parameter values (70) into the remote device (60) by the user interface (61), wherein the setting parameter values define a setting of the medical apparatus and are related to an identified patient and part of a patient prescription (73) for said identified patient;
generating, by a signal generator, a setting signal encoding said setting parameter values (70), said setting signal being variable as a function of the setting parameter values entered and comprising at least one between an optical pattern (80) and a frequency related signal optionally based on a NFC or Bluetooth protocol;
the method further comprising an acquisition procedure comprising the steps of:
providing the medical apparatus with a reader (40);
reading the setting signal by relatively approaching the reader (40) of the medical apparatus to the remote device (60) (the communication systems require physical proximity to the reader);
acquiring said setting parameter values (70) through the reading step;
wherein the medical apparatus is set based on the values of said setting parameter values.

A 2^{nd} aspect refers to a method for providing setting parameter values to an extracorporeal blood treatment apparatus (1) to perform an extracorporeal blood treatment,
the method comprising a remote procedure comprising the steps of:
providing a remote device (60) comprising a user interface (61);
entering setting parameter values (70) into the remote device (60) by the user interface (61), wherein the setting parameter values define a setting of the extracorporeal blood treatment apparatus (1) and are related to an identified patient and are part of a patient prescription (73) for said identified patient, the setting parameter values comprising at least one of:
   a patient fluid removal rate (PFR) for the identified patient; and
   two parameters for the identified patient between total weight loss (WL), total treatment time (TT) and ultrafiltration flow rate (UFR);
generating a setting signal, variable as a function of the setting parameter values entered, encoding said setting parameter values (70), said setting signal comprising at least one between an optical pattern (80) and a frequency related signal optionally based on a NFC or Bluetooth protocol (or based on any physical proximity wireless protocols);
the method further comprising an acquisition procedure comprising the steps of:
providing the extracorporeal blood treatment apparatus (1) with a reader (40);
reading the setting signal by relatively approaching the reader (40) of the extracorporeal blood treatment apparatus (1) to the remote device (60);
acquiring said setting parameter values (70) through the reading step;
wherein the extracorporeal blood treatment apparatus (1) is set based on the values of said setting parameter values.

A 3^{rd} aspect refers to a method (1000) for providing setting parameter values to an extracorporeal blood treatment apparatus (1) to perform an extracorporeal blood treatment, the method comprising a remote procedure comprising the steps of:
providing a remote device (60) comprising a user interface (61);
entering setting parameter values (70) into the remote device (60) through the user interface (61), wherein the setting parameter values define a setting of the extracorporeal blood treatment apparatus (1) and are related to an identified patient and are part of a patient prescription (73) for said identified patient, the setting parameter values (70) comprising at least one of:
   a patient fluid removal rate (PFR) for the identified patient; and
   two parameters for the identified patient between total weight loss (WL), total treatment time (TT) and ultrafiltration flow rate (UFR);
generating an optical pattern (80) encoding said setting parameter values (70), said optical pattern (80) being variable as a function of the setting parameter values entered;
the method further comprising an acquisition procedure comprising the steps of:
providing the extracorporeal blood treatment apparatus (1) with a reader (40);
displaying the optical pattern (80) on a display (62),
reading the optical pattern (80) displayed on the display (62) by relatively approaching the reader (40) of the extracorporeal blood treatment apparatus (1) to the display (62);
acquiring said setting parameter values (70) through the reading step;
wherein the extracorporeal blood treatment apparatus (1) is set based on the values of said setting parameter values.

A 5^{th} aspect refers to a system comprising medical apparatus (1) and a remote device (60), the medical apparatus (1) comprising:
a (proximity) reader (40) for reading optical patterns (80) and/or a frequency related signal;
a control unit (50) connected to the reader (40);
wherein the remote device (60) comprises a user interface (61), a signal generator and a controller (51) configured to:
   allowing entry of values for setting parameter values (70) via the user interface (61), wherein the setting parameter values define a setting of the medical apparatus (1) and are related to an identified patient, said setting parameter values being part of a patient prescription (73) for said identified patient,
   generating, by a signal generator of the remote device (60), a setting signal encoding said setting parameter values (70), said setting signal being variable as a function of the setting parameter values entered and comprising at least one between an optical pattern (80) and a frequency related signal optionally based on a NFC or Bluetooth protocol;
wherein the remote device (60) is a portable device generating said setting signal,
the control unit (50) of the medical apparatus (1) being configured to:
   acquire said setting parameter values (70) by reading the optical pattern (80) through the reader (40);
   setting the medical apparatus (1) based on the values of said setting parameter values.

In 6^{th} aspect according to the preceding aspects, wherein the remote device (60) is a portable device comprising both an input device (63) to enter the setting parameter values, and the signal generator, in particular one between the display and the frequency emitter, in particular the remote device (60) defining a single portable body.

In 7^{th} aspect according to the preceding aspects:
the setting signal is an optical pattern (80), the acquisition procedure comprising the steps of:
   displaying said optical pattern (80) on a display (62) of the remote device (60), and
   reading the optical pattern (80) by relatively approaching the reader (40) of the medical apparatus to the display (62) of the remote device (60); or
the setting signal is a frequency related signal, the acquisition procedure comprising the steps of:
   emitting said frequency related signal by a frequency emitter of the remote device (60), and
   reading the frequency related signal by relatively approaching the reader (40) of the medical apparatus to the display (62) of the remote device (60).

In a further aspect 7bis according to any one of the preceding aspects, the method comprises performing the remote procedure in a remote time interval, and performing the acquisition procedure in an acquisition time interval, wherein the acquisition time interval is shorter (e.g., much shorter) than the remote time interval.

In a further aspect 7ter according to the preceding aspect, the acquisition time interval lasts between 0.1 second and 40 seconds, more in particular between 1 second and 20 seconds, more in particular between 2 seconds and 10 seconds.

In a further aspect 7quater according to any one of the two preceding aspects, wherein the remote time interval lasts more than 1 minute (e.g., between 1 minute and 30 minutes), more in particular lasts more than 1.5 minutes (e.g., between 1.5 minutes 20 minutes), more in particular lasts more than 2/3 minutes (e.g., between 2/3 minutes and 5 minutes).

In a further aspect 7quinquies according to any one of the three preceding aspects, the acquisition time interval is at least the 30% shorter than the remote time interval, in particular the 50% shorter, more in particular the 80% shorter, more in detail the 90% shorter.

In a further aspect 7sexies according to any one of the four preceding aspects, the remote time interval is defined as an average time period necessary for a user, i.e. a physician or a nurse, to enter all the setting parameters of the medical apparatus to be able to perform the related medical treatment or of the extracorporeal blood treatment apparatus to be able to perform the related extracorporeal blood treatment.

In a further aspect 7septies according to any one of the five preceding aspects, the acquisition time interval is defined as a time period necessary, in particular to the control unit (50) of the apparatus (1), to perform at least the following steps of the acquisition procedure:
reading the setting signal by relatively approaching the reader (40) of the medical apparatus to the remote device (60); and
acquiring said setting parameter values (70) through the reading step;
optionally setting the apparatus (1) based on said setting parameter values (70).

An 8^{th} aspect refers to a system comprising an extracorporeal blood treatment apparatus (1) and a remote device (60), the extracorporeal blood treatment apparatus (1) comprising:
a filtration unit (2) having a primary chamber (3) and a secondary chamber (4) separated by a semi-permeable membrane (5);
a blood circuit comprising:
   a blood withdrawal line (6) extending between a first end connected to an inlet of the primary chamber and a second end for connection to a patient (P); and
   a blood return line (7) extending between a first end connected to an outlet of the primary chamber (3) and a second end for connection to said patient (P);
a blood pump (10) to circulate blood in the blood circuit;
an effluent line (12) connected to an outlet of the secondary chamber (4);
a reader (40) for reading optical patterns (80);
a control unit (50) for controlling the blood pump (10) and connected to the reader (40);
wherein the remote device (60) comprises a user interface (61) and a controller (51) configured to:
   allowing entry of values for setting parameter values (70) via the user interface (61), wherein the setting parameter values define a setting of the extracorporeal blood treatment apparatus (1) and are related to an identified patient, said setting parameter values being part of a patient prescription (73) for said identified patient, the setting parameter values (70) comprising at least one of:
      a patient fluid removal rate (PFR) for the identified patient; and
      two parameters for the identified patient between total weight loss, total treatment time and ultrafiltration flow rate;
   generating an optical pattern (80) encoding said setting parameter values (70), said optical pattern (80) being variable as a function of the setting parameter values entered;
wherein the control unit (50) of the extracorporeal blood treatment apparatus (1) is configured to:
   acquire said setting parameter values (70) by reading the optical pattern (80) through the reader (40);
   setting the extracorporeal blood treatment apparatus (1) based on the values of said setting parameter values.

In a 9^{th} aspect according to any one of the preceding aspects, the total weight loss parameter is a function of both the total treatment time and the ultrafiltration flow rate, in particular wherein:

$$W L = U F R \cdot T T$$

being:
WL=total weight loss;
UFR=ultrafiltration flow rate;
TT=total treatment time.

In a 10^{th} aspect according to any one of the preceding aspects, the optical pattern (80) comprises an image encoding information comprising said setting parameter values (70), in particular wherein the image encoding information comprises at least one between a bar code, a QR code, a steganographic image.

In an 11^{th} aspect according to any one of the preceding aspects, the acquisition procedure comprises a step of approaching, and in particular facing the display (62) of, the remote device (60) to the reader (40) of the medical apparatus.

In a 12^{th} aspect according to any one of the preceding aspects, the remote device (60) is a portable device defining one body and physically comprising both input device (63) to allow a user to enter the setting parameter values (70), and the display (62) to visualize the optical pattern (80).

In a 13^{th} aspect according to any one of the preceding aspects, the remote device (60) is a portable device defining one body and physically comprising both input device (63) to allow a user to enter the setting parameter values (70), and the emitter to emit the frequency related signal.

In a 14^{th} aspect according to any one of the preceding aspects, the setting parameter values (70) comprise a plurality of instructions for the extracorporeal blood treatment apparatus (1) to subsequently perform the extracorporeal blood treatment on the patient.

In a 15^{th} aspect according to any one of the preceding aspects, the setting parameter values (70) comprise a plurality of instructions for the medical apparatus (1) to subsequently perform the medical treatment on the patient.

In a 16^{th} aspect according to any one of the preceding aspects, the setting parameter values (70) further comprise at least one between a patient information (71) and a therapy configuration (72) of the extracorporeal blood treatment apparatus (1).

In a 17^{th} aspect according to any one of the preceding aspects, the setting parameter values (70) further comprise at least one between a patient information (71) and a therapy configuration (72) of the medical apparatus (1).

In a 18^{th} aspect according to any one of the preceding aspects, the patient information (71) comprises at least one information between a patient age, a patient weight, a patient haematocrit value, patient identification, a patient gender and a patient related disease.

In a 19^{th} aspect according to any one of the preceding aspects, the patient information (71) comprises a patient identification, in particular the name or an identification code of the patient.

In a 20^{th} aspect according to any one of the preceding aspects, the therapy configuration (72) comprises at least one information between:
a dialysis treatment type, comprising one of an ultrafiltration treatment (UF), a haemodialysis treatment (HD), a haemofiltration treatment (HF), haemodiafiltration treatment (HDF);
a disposable fluid line set type, optionally representative of the dialysis treatment type;
a dialysis filter or filter-set type representing a dialyzer type or a disposable set including the dialyzer type;
an anticoagulation setting for the extracorporeal blood treatment apparatus (1), in particular the anticoagulation being selected among systemic (e.g., heparin) anticoagulation and regional anticoagulation, for example citrate anticoagulation; and
presence or absence of a blood warmer.

In a 21^{st} aspect according to any one of the preceding aspects, the therapy configuration (72) comprises a dialysis treatment type, comprising one of an ultrafiltration treatment (UF), a haemodialysis treatment (HD), a haemofiltration treatment (HF), haemodiafiltration treatment (HDF).

In a 22^{nd} aspect according to any one of the preceding aspects, the therapy configuration (72) comprises a disposable fluid line set type, optionally representative of the dialysis treatment type.

In a 23^{rd} aspect according to any one of the preceding aspects, the therapy configuration (72) comprises a dialysis filter or filter-set type representing a dialyzer type or a disposable set including the dialyzer type.

In a 24^{th} aspect according to any one of the preceding aspects, the therapy configuration (72) comprises an anticoagulation setting for the extracorporeal blood treatment apparatus (1), in particular the anticoagulation being selected among systemic (e.g. heparin) anticoagulation and regional anticoagulation, for example citrate anticoagulation.

In a 25^{th} aspect according to any one of the preceding aspects, the therapy configuration (72) comprises an indication of presence or absence of a blood warmer.

In a 26^{th} aspect according to any one of the preceding aspects, the patient prescription (73) comprises one or more flow rate values to be set to the extracorporeal blood treatment apparatus (1), the flow rate being selected between a blood flow rate, a Pre Blood Pump (PBP) flow rate, a syringe flow rate, a fresh dialysis flow rate and a replacement fluid flow rate;
in particular the one or more flow rates of the patient prescription are a function a therapy configuration (72), for example function of a dialysis treatment type and/or and disposable fluid line set type.

In a 27^{th} aspect according to the preceding aspect, the flow rate values comprise the Pre Blood Pump (PBP) flow rate and the fresh dialysis flow rate.

In a 28^{th} aspect according to the preceding two aspects, the flow rate values comprise the replacement fluid flow rate.

In a 29^{th} aspect according to any one of the preceding aspects, the method further comprises:
providing at least one disposable component for the extracorporeal blood treatment apparatus (1) necessary for a treatment to be carried out;
providing the apparatus with an identification code of said at least one disposable component to identify the disposable component;
and wherein the method further comprises a consistency procedure comprising the steps of:
verifying consistency between the identified disposable component and the setting parameter values acquired through reading the optical image from the display;
if at least one setting parameter is not consistent with the identified disposable component for the extracorporeal blood treatment apparatus (1), preventing the extracorporeal blood treatment apparatus (1) to be set based on said setting parameter values.

In a 30^{th} aspect according to any one of the preceding aspects, the medical apparatus, in particular the extracorporeal blood treatment apparatus, is configured for:
receiving at least one disposable component for the extracorporeal blood treatment apparatus (1) necessary for a treatment to be carried out;
receiving an identification code of said at least one disposable component to identify the disposable component;
and wherein the control unit (50) of the medical apparatus is configured to perform a consistency procedure comprising the steps of:
verifying consistency between the identified disposable component and the setting parameter values acquired through reading the optical image from the display;
if at least one setting parameter is not consistent with the identified disposable component for the extracorporeal blood treatment apparatus (1), preventing the extracorporeal blood treatment apparatus (1) to be set based on said setting parameter values.

In a 31^{st} aspect according to any one of the preceding aspects, the consistency procedure includes the step of reading said identification code by:
the reader (40) of the medical apparatus, in particular of the extracorporeal blood treatment apparatus (1), to identify the disposable component, said identification code of said at least one disposable component being in the form of a respective optical pattern (80); or
a frequency scanner, e.g., distinct from the reader (40) of the medical apparatus, to identify the disposable component, said identification code of said at least one disposable component being in the form of a frequency related signal (such as and RFID tag).

In a 32^{nd} aspect according to any one of the preceding aspects, each disposable component comprises a component identifier encoding said identification code, and the setting parameter values (70) comprise a treatment identifier representative of the dialysis treatment to be set in the extracorporeal blood treatment apparatus (1), in particular said dialysis treatment being one between ultrafiltration treatment (UF), a haemodialysis treatment (HD), a haemofiltration treatment (HF), haemodiafiltration treatment (HDF),
the consistency procedure comprising verifying whether said component identifier of the component matches the treatment identifier of the apparatus according to a predefined compatibility list, said predefined compatibility list between a series of component identifiers and a series of treatment identifiers,
in particular wherein the compatibility list comprises, for each dialysis treatment between ultrafiltration treatment (UF), a haemodialysis treatment (HD), a haemofiltration treatment (HF), haemodiafiltration treatment (HDF), a series of identification codes representatives of compatible disposable components.

In a 33^{rd} aspect according to any one of the preceding aspects, the consistency procedure further comprises the steps of:
defining a respective threshold range for one or more of the setting parameter values (70) based on the identification code of the disposable component, in particular defining a respective threshold range, based on the identification code of the component, for at least one of:
   the patient fluid removal rate (PFR) for the identified patient; and
   two parameters for the identified patient between total weight loss (WL), total treatment time (TT) and ultrafiltration flow rate (UFR);
if at least one setting parameter value exceeds the respective threshold range, preventing the extracorporeal blood treatment apparatus (1) to be set based on said setting parameter values.

In a 34^{th} aspect according to the preceding aspect, the respective threshold range is referred to a respective value related to at least one of the patient information (71), wherein the method defines, or the control unit (50) of the medical apparatus is configured to define, a threshold range relative to the weight and/or age of the patient based on the identification code of the disposable component mounted on the apparatus, and wherein, if the actual weight and/or age of the patient exceeds the respective threshold range, the consistency procedure prevents the extracorporeal blood treatment apparatus (1) to be set based on said setting parameter values.

In a 35^{rd} aspect according to any one of the preceding aspects, the setting parameter values comprise patient information (71) including an identification of the patient, in particular the name, identifying the patient which the setting parameter values relate to, the method further comprising, or the control unit of the medical apparatus being configured to perform, a cross check procedure including the steps of:
associating the extracorporeal blood treatment apparatus (1) with a patient intended to undergo the extracorporeal blood treatment;
providing the medical apparatus, in particular the extracorporeal blood treatment apparatus (1), with an identification of said patient from an information source different from the optical pattern (80), in particular providing the extracorporeal blood treatment apparatus (1) with the name of said patient;
checking the match between said patient identification of the setting parameter values read through the optical pattern (80) and the identification related to said patient provided through the information source different from the optical pattern (80);
if no match is found between the patient identification of the setting parameter values and the identification related to said patient from the information source, preventing the medical apparatus (1), in particular the extracorporeal blood treatment apparatus (1), to be set based on said setting parameter values and optionally issuing an alarm, i.e. an acoustic or visible alarm.

In a 36^{th} aspect according to any one of the preceding aspects, the step of providing the medical apparatus, in particular to the extracorporeal blood treatment apparatus (1), with information related to said person comprises the steps of:
providing said patient with an identification card or an identification wearable device, e.g., an identification bracelet;
providing the extracorporeal blood treatment apparatus (1) with a scanner;
scanning the identification card or the identification wearable device by said scanner of the extracorporeal blood treatment apparatus (1) to provide the medical apparatus, in particular of the extracorporeal blood treatment apparatus (1), with the information related to identification of said patient,
wherein the identification card or the identification wearable device comprises at least one between an optical pattern (80), such as a QR code or a bar-code, and a Near Field Communication NFC device or an RFID, optionally said scanner of the extracorporeal blood treatment apparatus (1) coinciding with the reader (40) of the extracorporeal blood treatment apparatus (1).

In a 37^{th} aspect according to the preceding aspect, the medical apparatus, in particular the extracorporeal blood treatment apparatus, comprises the scanner configured to scan the identification card or the wearable device, for example the identification bracelet, to obtain information related to identification said patient.

In a 38^{th} aspect according to any one of the preceding aspects, the remote device (60) is distinct and physically separated by the medical apparatus or by extracorporeal blood treatment apparatus (1).

In a 39^{th} aspect according to any one of the preceding aspects the acquisition procedure includes a safety procedure comprising a step of verifying a validity of the setting parameter values acquired by the optical pattern (80), in particular said safety procedure comprising verifying whether all the setting parameter values are within a predefined validity range, if one or more of said setting parameter values exceeds said validity range, the safety procedure prevents the medical apparatus or the extracorporeal blood treatment apparatus (1) to be set based on said setting parameter values.

In a 40^{th} aspect according to the preceding aspect, if one or more of said setting parameter values exceeds said validity range, the safety procedure includes commanding the medical apparatus or the extracorporeal blood treatment apparatus (1) to emit an alarm signal, such as an acoustic alarm signal or an alarm displayed on a display (62) of the extracorporeal blood treatment apparatus (1).

In a 40^{th} bis aspect according to any one of the preceding aspects, the acquisition procedure comprises a confirmation procedure temporally subsequent the step of acquiring the setting parameter values (70) by reading the optical pattern (80) through the reader (40), in particular the confirmation procedure being performed by the apparatus (1),
wherein the confirmation procedure comprises the steps of:
   displaying on a display, in particular a display of the apparatus (1), one or more of the setting parameter values (70);
   requiring to receive from a user an approval signal of said one or more of the setting parameter values (70) received by the apparatus (1);
   checking whether the approval signal is received;
in particular wherein:
   if the approval signal is properly received by the apparatus (1), allowing the step of setting the extracorporeal blood treatment apparatus (1) based on the values of said setting parameter values (70) received by the apparatus;
   if the approval signal is not properly received by the apparatus (1), preventing the step of setting the extracorporeal blood treatment apparatus (1) based on the values of said setting parameter values (70) received by the apparatus

In a 40^{th} ter aspect according to any one of the preceding aspects, the requiring step of the confirmation procedure comprises the step of:
requiring one and only one approval signal representative of approval of all the setting parameter values (70) received by the apparatus (1); or
partitioning the setting parameter values (70) into two or more groups each one comprising respective setting parameter values (70) received by the apparatus (1), and requiring an approval signal for each group of setting parameter values (70);
requiring, for each setting parameter value (70) of the setting parameter values (70), a respective approval signal representative of approval of said setting parameter value (70) received by the apparatus (1).

In a 40^{th} quater aspect according to the preceding aspect, the setting parameter values (70) are partitioned in said groups based on a category each setting parameter value, wherein at least a flow rate group is provided comprising only the setting parameter values related to a flow rate to set the extracorporeal blood treatment apparatus (1),
optionally wherein an identification group is provided comprising only the setting parameter values related to identification of the patient,
optionally wherein a therapy group is provided comprising only the setting parameter values related to the therapy configuration (72).

In a 40^{th} quinquies aspect according to the preceding aspect, the confirmation procedure comprises providing at least one between the flow rate group, the identification group and the therapy group.

In a 41^{st} aspect according to any one of the preceding aspects, the method comprises a step of pairing the remote device (60) with the medical apparatus or the extracorporeal blood treatment apparatus (1) based on an authentication code to allow an authorized user to perform the pairing step, so that:
if the pairing step is successful, the method performs the acquisition procedure;
if the pairing step is not successful, the method prevents the acquisition procedure to be carried on, or the method prevents the setting parameter values to set the extracorporeal blood treatment apparatus (1), optionally said pairing being based on NFC or Bluetooth protocols.

In a 42^{nd} aspect according to any one of the preceding aspects, the control unit (50) of the medical apparatus and the controller (51) of the remote device are configured to perform a step of pairing the remote device (60) with the medical apparatus, in particular with the extracorporeal blood treatment apparatus (1), based on an authentication code to allow an authorized user to perform the pairing step, so that:
if the pairing step is successful, the control unit (50) of the medical apparatus and the controller (51) of the remote device perform the acquisition procedure;
if the pairing step is not successful, the control unit (50) of the medical apparatus and the controller (51) of the remote device prevent the acquisition procedure to be carried on, or prevent the setting parameter values to set the medical apparatus, in particular the extracorporeal blood treatment apparatus (1), optionally said pairing being based on NFC or Bluetooth protocols.

In a 43^{rd} aspect according to any one of the preceding aspects, the authentication code comprises at least one between a password, a serial number, a fingerprint, a facial recognition algorithm.

In a 44^{th} aspect according to any one of the preceding aspects, the method includes, or the control unit (50) and the controller (51) are configured for, pairing the remote device (60) with the medical apparatus, in particular with the extracorporeal blood treatment apparatus (1), every time the setting parameter values are acquired and read by the reader (40).

In a 45^{th} aspect according to any one of the preceding aspects, the optical pattern (80) which encodes the setting parameter values further includes the authentication code to allow pairing of the remote device (60) with the apparatus, in particular with the extracorporeal blood treatment apparatus (1).

In a 46^{th} aspect according to any one of the preceding aspects, a variation of the setting parameter values entered corresponds to a variation in the optical pattern (80) displayed on the user interface (61) of the remote device (60).

In a 47^{th} aspect according to any one of the preceding aspects, the remote device (60) comprises a case, the display 62, a battery, input device (63) to allow a user to enter the setting parameter values, and the controller (51) operatively connected at least to the display 62, the battery, and the input device (63), optionally wherein the display (62) and the input device (63) define a touchscreen or wherein the input device (63) is a keyboard (for example the remote device may be a mobile phone or a tablet).

In a 48^{th} aspect according to any one of the preceding aspects, the acquisition procedure is performed temporally after the remote procedure.

In a 49^{th} aspect according to any one of the preceding aspects, said setting parameter values are necessary for the extracorporeal blood treatment apparatus (1) to perform the dialysis treatment.

In a 50^{th} aspect according to any one of the preceding aspects, said setting parameter values are necessary for the medical apparatus (1) to perform the medical treatment.

In a 51^{st} aspect according to any one of the preceding aspects, the remote procedure is performed at a remote distance from the patient and/or from the medical apparatus, while the acquisition procedure is performed at an acquisition distance from the patient and/or from the medical apparatus, wherein the remote distance is longer than the acquisition distance, optionally wherein the medical apparatus is the extracorporeal blood treatment apparatus.

Usually the remote procedure is performed in a different room with respect to the acquisition procedure.

In a 52^{nd} aspect according to any one of the preceding aspects, the remote procedure is performed:
remotely at a safety distance with respect to the patient and/or with respect to the medical apparatus, in particular the safety distance being comprised between 2 meters and 100 meters, in particular between or 3 meters and 50 meters, more in particular between 4 meters and 20 meters; and/or
separately from the patient by a barrier, for example one between a wall, a window, a transparent wall, wherein said barrier and/or said safety distance being configured to isolate a user, in charge of entering the setting parameter values (70) into the remote device (60), from the patient to avoid bacteria and/or virus transmission between the patient and the user, optionally wherein the medical apparatus is the extracorporeal blood treatment apparatus.

In a 53^{rd} aspect according to any one of the preceding aspects, the acquisition procedure is performed close to the medical apparatus, in particular close to the extracorporeal blood treatment apparatus (1), and close to the patient, wherein during the acquisition procedure the user, in charge of performing the acquisition procedure, is at a distance from the patient shorter than 10 meters, in particular shorter than 5 meters, more in particular shorter than 3 meters, more in particular shorter than 1 meter.

In a 54^{th} aspect according to any one of the preceding aspects, the cross check procedure (400) is performed at a cross check distance substantially equal to the acquisition distance.

In a 55^{th} aspect according to any one of the preceding aspects, the user interface (61) of the remote device (60) comprises both the display (62) and an input device (63) for entering the setting parameter values, the method further comprising a local procedure, temporally interposed between the remote procedure and the acquisition procedure, comprising the steps of:
entering one or more further setting parameter values (70) into the remote device (60) through the user interface (61) of the remote device (60), said further setting parameter values comprising one or more of the setting parameter values in particular at least one between a patient prescription (73), a therapy configuration (72) and a patient information (71); and/or
modifying or updating, through the user interface (61) of the remote device (60), one or more of the setting parameter values (70) entered during the remote procedure, wherein the local procedure is performed by a user at a local distance from the patient and/or from the medical apparatus, while the remote procedure is performed by a user at a remote distance from the patient and/or from the medical apparatus, wherein the local distance is shorter than the remote distance, optionally the local distance being shorter than 10 meters, in particular shorter than 5 meters, more in particular shorter than 3 meters, more in particular shorter than 1 meter.

In a 56^{th} aspect according to any one of the preceding aspects, the user interface (61) of the remote device (60) comprises both the display (62) and an input device (63) for entering the setting parameter values, and wherein the controller (51) of remote device (60) is configured to perform a local procedure, temporally interposed between the remote procedure and the acquisition procedure, comprising the steps of:
allowing a user to enter one or more further setting parameter values (70) into the remote device (60) through the user interface (61) of the remote device (60), said further setting parameter values comprising one or more of the setting parameter values in particular at least one between a patient prescription (73), a therapy configuration (72) and a patient information (71); and/or
allowing a user to modify or update, through the user interface (61) of the remote device (60), one or more of the setting parameter values (70) entered during the remote procedure, wherein the local procedure is performed by a user at a local distance from the patient and/or from the medical apparatus, while the remote procedure is performed by a user at a remote distance from the patient and/or from the medical apparatus, wherein the local distance is shorter than the remote distance, optionally the local distance being shorter than 10 meters, in particular shorter than 5 meters, more in particular shorter than 3 meters, more in particular shorter than 1 meter.

In a 57^{th} aspect according to any one of the preceding aspects, the local procedure and the acquisition procedure are performed substantially at the same place, in particular at the same distance with respect to the patient and/or to the medical apparatus.

In a 58^{th} aspect according to any one of the preceding aspects, the optical pattern (80) is displayed on the display (62) of the remote device (60), and wherein the step of displaying the optical pattern (80) on said display (62) is performed during the acquisition procedure and temporally after the remote procedure, in particular wherein the step of displaying the optical pattern (80) on the display (62) is subordinated to the successful pairing between the remote device (60) and the medical apparatus, in particular to the extracorporeal blood treatment apparatus (1), in particular wherein if the paring is not successful the method prevents the optical pattern (80) to be displayed.

In a 59^{th} aspect according to any one of the preceding aspects, the remote device (60) comprises a battery to supply the user interface (61), in particular the display (62) and the input device (63) of the user interface (61).

In a 60^{th} aspect according to any one of the preceding aspects, the remote device (60) is a portable device generating the setting signal, in particular generating the optical pattern and/or the frequency related signal encoding the setting parameter values (70).

In a 61^{st} aspect according to any one of the preceding aspects, the remote device (60) is a portable device comprising both an input device (63) to enter the setting parameter values, and the signal generator, in particular one between the display and the frequency emitter, in particular the remote device (60) defining a single portable body.

In a 62^{nd} aspect according to any one of the preceding aspects, the controller (51) of the remote device (60) is configured to perform the remote procedure (100).

In a 63^{rd} aspect according to any one of the preceding aspects, the control unit (50) of the medical apparatus (1) is configured to perform the acquisition procedure (200).

In a 64^{th} aspect according to any one of the preceding aspects, the control unit (50) of the medical apparatus (1) is configured to perform the consistency procedure (300).

In a 65^{th} aspect according to any one of the preceding aspects, the control unit (50) of the medical apparatus (1) is configured to perform the cross check procedure (400).

In a 66^{th} aspect according to any one of the preceding aspects, the control unit (50) of the medical apparatus (1) is configured to perform the safety procedure (500).

In a 67^{th} aspect according to any one of the preceding aspects, the control unit (50) of the medical apparatus (1) and the controller (51) of the remote device are configured to perform the pairing step (600).

In a 68^{th} aspect according to any one of the preceding aspects, the controller (51) of the remote device (60) is configured to perform the local procedure (700).

In a 69^{th} aspect according to any one of the preceding aspects, the step of displaying the optical pattern (80) on the display (62) of the remote device (60) is part of the remote procedure or of the acquisition procedure.

In a 70^{th} aspect according to any one of the preceding aspects, the controller (51) of the remote device (60) is configured to perform the step of displaying the optical pattern (80) on the display (62) of the remote device (60).

In a 70^{th} aspect according to any one of the preceding aspects, the controller (51) of the remote device (60) is configured to emit the setting signal in the form of the optical pattern (80) on the display (62) of the remote device (60) or in the form of a frequency related signal such as based on NFC, RFid or radio frequency technology.

### DRAWINGS

Some embodiments and some aspects of the invention will be described below with reference to the attached drawings, provided for illustrative purposes only, wherein:

FIG. 1 is a schematic view of an extracorporeal blood treatment apparatus;

FIGS. 2 and 3 are schematic views of dialysis and blood circuits for a chronic dialysis treatment;

FIG. 4 is a schematic view of a remote device having an user interface showing setting parameter values related to patient information;

FIG. 5 is a schematic view of a remote device having an user interface showing setting parameter values related to a therapy configuration;

FIG. 6 is a schematic view of a remote device having an user interface showing setting parameter values related to a patient prescription;

FIG. 7 is a schematic view of a remote device having a display visualizing an optical pattern encoding the setting parameters;

FIG. 8 is a schematic view of a remote device approached to the medical apparatus during the acquisition procedure;

FIG. 9 is a flow chart comprising steps of the setting method according to an embodiment of the present invention;

FIG. 10 is a flow chart comprising steps of the setting method according to a further embodiment of the present invention.

### DEFINITIONS

In this detailed description, corresponding parts illustrated in the various figures are indicated with the same numerical references. The figures may illustrate the invention by means of non-scale representations; therefore, parts and components illustrated in the figures relating to the object of the invention may relate exclusively to schematic representations.

Upstream and/Downstream

The terms upstream and downstream refer to a direction or trajectory of advancement of a fluid configured to flow within the fluid lines of the circuit of the apparatus during normal use of the apparatus (e.g., during treatment).

### DETAILED DESCRIPTION

A setting method 1000 and a related system 90 for setting a medical apparatus are described here after in detail. The present setting method 1000 and related system 90 may be performed on an extracorporeal blood treatment apparatus 1: in more detail, the latter may be an extracorporeal membrane oxygenation apparatus, a dialysis apparatus for performing treatments such as an ultrafiltration treatment UF, a haemodialysis treatment HD, a haemofiltration treatment HF, or a haemodiafiltration treatment HDF. The dialysis apparatus may be directed to chronic or acute treatments. Alternatively, the present method and related system may be performed on a peritoneal dialysis apparatus. Moreover, the present method and related system may be implemented on an infusion pump.

### Setting Method 1000

The setting method 1000 is directed to provide setting parameter values 70 to a medical apparatus to perform a medical treatment. The expression “medical apparatus” may refer to a number of different medical apparatus which need a setting procedure to start a proper medical treatment related to an identified patient. The setting procedure is patient-related according to a patient prescription 73 provided, for example, by a clinician.

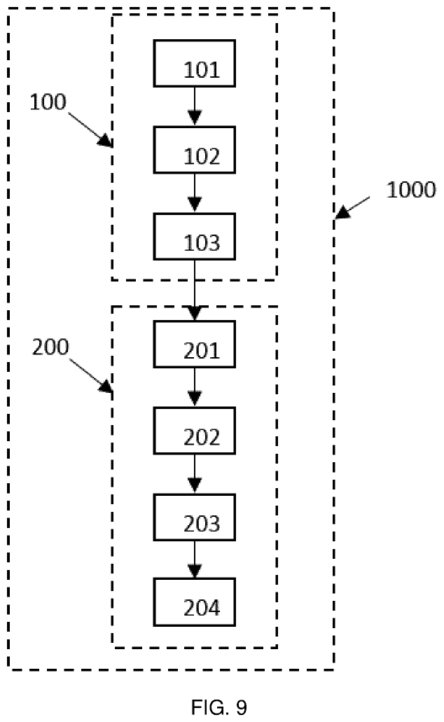

The method 1000 comprises a remote procedure 100, as in FIG. 9, comprising the steps of providing 101 a remote device 60 comprising a user interface 61, entering 102 setting parameter values 70 into the remote device 60 by the user interface 61, and generating 103, by a signal generator, a setting signal encoding the setting parameter values 70, wherein the setting signal is variable as a function of the setting parameter values 70 entered. A variation in the setting parameter values 70 corresponds to a variation in the setting signal.

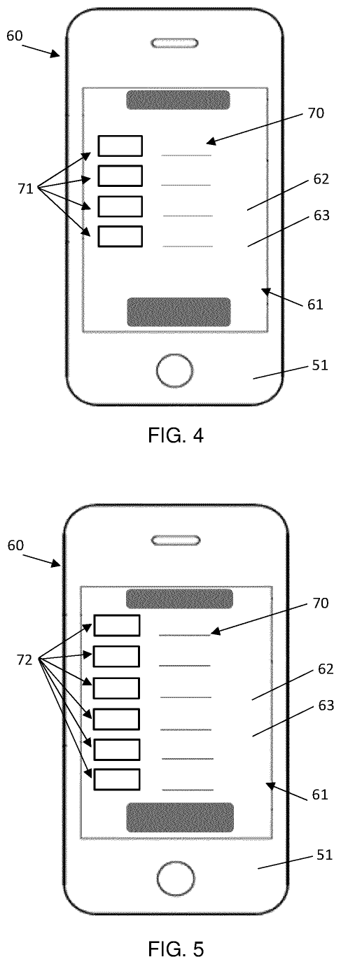

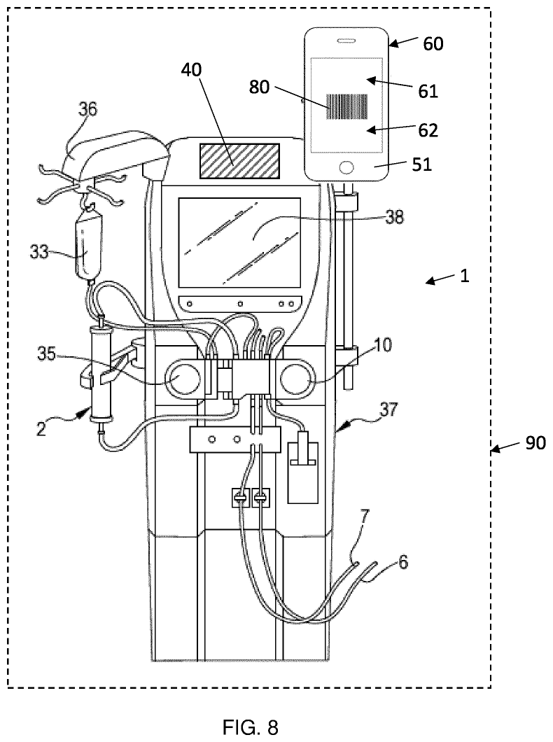

The remote device 60 may be a smartphone, a tablet or an electronic tool to allow the user to enter the setting parameter values 70, as shown in FIGS. 4-8: on this regard, the user interface 61 may include an input device 63, such as a keyboard, a mouse or a touchscreen, to allow the user to type or select the setting parameter values 70. In particular, the remote device 60 may be a hand-portable device having dimensions and weight compatible with easy transportation, i.e. to allow the user to keep the remote device 60 by hand.

Notably, the remote device 60 and the medical apparatus, such as the extracorporeal blood treatment apparatus, are separate and distinct each other. In particular, the term “remote” indicates that the remote device 60 is separated by the medical apparatus 1 and potentially usable far from the medical apparatus 1 to enter the setting parameter values 70. The patient is supposed to be located at or very close to the medical apparatus. For example, the user, i.e. a nurse, may keep the remote device 60 by hand far from the medical apparatus 1, i.e. at a distance which would prevent the nurse to directly operate on the medical apparatus: at this stage, the nurse enters the setting parameter values 70 into the remote device 60 through the input device 63 of the user interface 61 of the remote device 60, keeping herself far for the patient. Then the nurse commands the remote device 60 to generate, by the signal generator, the setting signal encoding the setting parameter values 70.

The setting signal may comprise an optical pattern 80, such as a bar code, a QR code, a steganographic image or the like, as shown in FIG. 7. The optical pattern 80 may be in white and black, grey scale or may comprise colors, i.e. RGB colors. Thus the step of generating 103 the setting signal comprises the step of generating the optical pattern on a display, i.e. on a display 62 of the remote device. Alternatively, or in addition, the setting signal may comprise a frequency related signal, such as based on Near Field Communication NFC or an RFid communication technology. Thus the step of generating 103 the setting signal comprises the step of generating the frequency related signal by an emitter of the remote device.

The setting parameter values 70 are encoded in the bar code, QR code, steganographic image, NFC, or RFid signal. A variation in the setting parameter values 70 corresponds to a variation in the optical pattern 80 or in the frequency related signal.

The setting parameter values 70 define a setting of the medical apparatus and are related to an identified patient and are part of a patient prescription 73 for the identified patient. In particular the setting parameter values 70 comprise a plurality of instructions for the extracorporeal blood treatment apparatus to subsequently perform the medical treatment on the patient, such as the extracorporeal blood treatment. As the setting parameter values 70 define a setting of the medical apparatus, these parameters are necessary for the medical treatment to be carried out. In particular, the way the treatment is carried out is related to the patient prescription 73. Notably, the setting parameter values 70 are strictly related to the identified patient.

The remote procedure 100 may be performed remotely at a safety distance with respect to the patient and/or with respect to the medical apparatus 1: in particular the remote procedure 100 may be performed at a safety distance between 2 meters and 100 meters, in particular between or 3 meters and 50 meters, more in particular between 4 meters and 20 meters. The remote procedure may be performed separately from the patient by a barrier: in particular the user in charge of performing the remote procedure is separated from the patient and/or from the medical apparatus 1 by this barrier which may be a wall, a window, or a transparent wall. In more detail, this barrier and this safety distance are configured to isolate the user, in charge of entering the setting parameter values 70 into the remote device 60, from the patient to avoid bacteria and/or virus transmission between the patient and the user.

The setting parameter values 70 are part of a patient prescription 73 and comprise at least one between a patient fluid removal rate PFR for the identified patient, and two parameters for the identified patient between total weight loss WL, total treatment time TT and ultrafiltration flow rate UFR. Notably, the patient fluid removal rate PFR corresponds to the fluid rate removed from the patient during acute dialysis treatments (e.g., in intensive care units). In acute treatments, total treatment time is not provided since the patient may remain under treatment for a prolonged period of time such as days, weeks or even a longer time. Therefore, the prescription would include a patient fluid removal rate, namely the fluid intended to be removed from the patient per unit of time. The patient fluid removal rate PFR may be dimensionally defined in terms of ml/min.

Differently, in chronic treatments, the patient undergoes a few sessions per week having a prefixed time duration. During each session a certain amount of fluid has to be removed primarily corresponding to the fluid overload gained in the time between consecutive sessions. The total weight loss WL corresponds to the weight of fluid removed from the patient during a chronic dialysis treatment. The total weight loss may be dimensionally defined in terms of liters or kg. Notably, the total weight loss WL, the total treatment time TT and ultrafiltration flow rate UFR are linked together by the following formula:

$$W L = U F R \cdot T T$$

wherein:
WL=total weight loss;
UFR=ultrafiltration flow rate;
TT=total treatment time.

Thus, the total weight loss is a function of both the total treatment time and the ultrafiltration flow rate. The user may set the total weight loss and the total treatment time or alternatively may enter the ultrafiltration flow rate and the total treatment time which, combined according to the above formula, define the total weight loss. Two out of three parameters are set, thereby defining the missing one.

The patient prescription 73 may also comprise a number of flow rate values to be set to the extracorporeal blood treatment apparatus, such as a blood flow rate, a Pre Blood Pump PBP flow rate, a syringe flow rate, a fresh dialysis flow rate and a replacement fluid flow rate. The flow rates to be set depends on the selected treatment type. Usually (but not limitatively), PBP flow rate is set in case of regional anticoagulation procedures requiring a regional anticoagulant (e.g., citrate) to be infused into the blood upstream the blood pump. In such a case, generally the syringe flow rate may refer to infusion of a calcium solution (either into the venous return line or directly into the patient) to balance calcium loss in the filtration unit.

Alternatively, the syringe may inject heparin or other systemic anticoagulant into the blood withdrawal line.

The replacement fluid may comprise dialysis fluid infused in the blood circuit upstream and/or downstream the filtration unit in case of HF or HDF treatments.

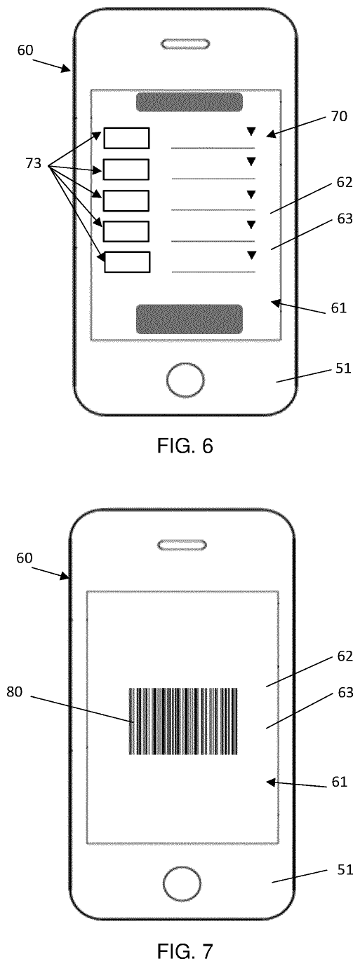

The method may comprise displaying the patient prescription 73 on the user interface 61, i.e. a display 62, of the remote device 60 to allow the user to enter the values relative to the patient prescription 73, such as the patient fluid removal rate PFR (acute field), or, alternatively, the total weight loss WL, the total treatment time TT and the ultrafiltration flow rate UFR, as shown in FIG. 6.

The setting parameter values 70 may also comprise a therapy configuration 72 comprising a dialysis treatment type, i.e. whether the medical treatment is an ultrafiltration treatment UF, a haemodialysis treatment HD, a haemofiltration treatment HF, or a haemodiafiltration treatment HDF.

The therapy configuration 72 may also comprise selection of a disposable fluid line set type, which may be representative of the dialysis treatment type, too. The disposable fluid line set type comprises a batch of fluid lines defining the blood circuit and optionally infusion lines: in particular the fluid lines may comprise the blood withdrawal line 6, the blood return line 7, and optionally the infusion line 34.

In case of an apparatus for acute treatment dialysis, the disposable fluid line set may also comprise fluid lines defining the dialysis circuit. Alternatively, in case of an apparatus for chronic treatment dialysis, the dialysis circuit is composed of permanent fluid lines which need to be primed and sterilized for each treatment.

Notably, each disposable fluid line set may substantially correspond to a specific dialysis treatment type: in particular the haemodiafiltration treatment HDF is associated to a corresponding disposable fluid line set.

Alternatively, a general purpose disposable fluid line set may be available allowing to perform a number of different treatments (e.g., UF, HD, and HDF). In more general terms, a certain disposable fluid line set allows to perform a plurality, but not all, dialysis treatment types.

Furthermore, the therapy configuration 72 may comprise a dialysis filter or filter-set type representing a dialyzer type or a disposable set including the dialyzer type, namely the filtration unit 2. The dialyzer type may vary as a function of patient info, such as the patient age, or as a function of the treatment type. For example, a dialyzer for ultrafiltration treatment UF may technically differ from a dialyzer for haemodiafiltration treatment HDF.

The therapy configuration 72 may further comprise an anticoagulation setting for the extracorporeal blood treatment apparatus: in particular the anticoagulation may be selected among heparin anticoagulation (systemic) and regional anticoagulation, for example citrate anticoagulation.

In addition, the therapy configuration 72 may indicate the presence (namely, the need) or absence of a blood warmer in the extracorporeal blood treatment apparatus.

The method may comprise displaying the therapy configuration 72 on the user interface 61, i.e. a display 62, of the remote device 60 to allow the user to enter values of the therapy configuration 72 as shown in FIG. 5: the user may enter values related to the therapy configuration 72 or indicate the treatment type, the filter type, the anticoagulation setting and/or the presence or absence of the blood warmer.

The setting parameter values 70 may also comprise patient information 71 including a patient identification, such as the name or an identification code to allow a user, i.e. a nurse, to identify the patient before starting the medical treatment. Notably, the therapy configuration 72 and the patient prescription 73 are strictly related to the patient: thus identification of the patient which has to undergo the treatment is fundamental to start the treatment. Furthermore, the patient information 71 may include a patient age, a patient weight, a patient hematocrit value, a patient gender and a patient related disease. The hematocrit value of the patient may be measured before starting the treatment and/or before defining the patient prescription 73, so that the latter may be set as a function of the hematocrit value.

The method may comprise displaying the patient information 71 on the user interface 61, i.e. a display 62, of the remote device 60 to allow the user to enter values of the patient information 71 as shown in FIG. 4: the user may enter values related to the patient information 71 such as the an identification of the patient.

The method further comprises an acquisition procedure 200, as in FIG. 9, comprising the steps of emitting 201 the setting signal encoding the setting parameter values 70, providing 202 the medical apparatus with a reader 40, reading 203 the setting signal by relatively approaching the reader 40 of the medical apparatus to the remote device 60, and acquiring 204 the setting parameter values 70 through the reading step. Then, the medical apparatus is set based on the values of the setting parameter values 70. Notably, the acquisition procedure 200 is performed temporally after the remote procedure 100, namely once the setting parameter values 70 have been entered into the remote device 60 by a user, i.e. by a nurse or a clinician. Furthermore, the acquisition procedure 200 is performed at an acquisition distance from the patient and/or from the medical apparatus 1, while the remote procedure 100 is performed at a remote distance from the patient and/or from the medical apparatus: the remote distance is longer than the acquisition distance. In other terms, the acquisition procedure is performed locally close to the patient and close to the medical apparatus 1 which performs the medical treatment on the patient.

During the acquisition procedure 200 the user, in charge of performing the acquisition procedure, is at an acquisition distance from the patient and/or from the medical apparatus shorter than 10 meters, in particular shorter than 5 meters, more in particular shorter than 3 meters, more in particular shorter than 1 meter. Notably, the medical apparatus may be the extracorporeal blood treatment apparatus 1 and the medical treatment may be the extracorporeal blood treatment, such as the dialysis treatment.

Furthermore, also the time requested to perform the acquisition procedure is much shorter than the time requested, i.e. to a physician/nurse, to perform the remote procedure. In more detail the method performs the remote procedure in a remote time interval, and performs the acquisition procedure in an acquisition time interval: the acquisition time interval is much shorter than the remote time interval. For example the acquisition time interval may last between 0.1 second and 40 seconds, more in particular between 1 second and 20 seconds, more in particular between 2 seconds and 10 seconds. On the other hand, the remote time interval may last between 1 minute and 30 minutes, more in particular between 1.5 minutes 20 minutes, more in particular between 2 minutes and 5 minutes.

Thus, the acquisition time interval may be at least the 30% shorter than the remote time interval, in particular the 50% shorter, more in particular the 80% shorter, more in detail the 90% shorter than the remote time interval. Notably, the remote time interval may be defined as an average time period necessary for a user, i.e. a physician/nurse, to enter all the setting parameters of the medical apparatus to be able to perform the related medical treatment, or of the extracorporeal blood treatment apparatus to be able to perform the related extracorporeal blood treatment. This time period may take into account the time for typing on a keyboard all the setting parameter values.

On the other hand, the acquisition time interval is defined as a time period necessary, in particular for the control unit 50 of the apparatus 1, to perform at least the following steps of the acquisition procedure:
reading the setting signal by relatively approaching the reader 40 of the medical apparatus to the remote device 60; and
acquiring said setting parameter values 70 through the reading step;
optionally setting the apparatus 1 based on said setting parameter values 70.

Notably, based on the fact that the acquisition procedure is performed by an electronic control unit, namely the control unit 50, and based on that the entering step of the remote procedure is performed manually by an operator, the acquisition time interval is by nature mush shorter than the remote time interval.

The fact that the acquisition time interval is much shorter than the remote time interval allows the operator, i.e. the nurse, to reduce the time spent close to the patient, thereby reducing the risks being exposed to contagious diseases of the patient.

The step of emitting the setting signal may comprise displaying 201 the optical pattern 80 encoding the setting parameter values 70 on a display. The display may be the display 62 of the remote device 60, so that the remote device 60 comprises in a single body the input device 63 to allow a user to insert the setting parameter values 70, and the display 62 to visualize the optical pattern 80 encoding the setting parameter values 70. The Display 62 may be a LCD, LED screen or any other suitable electronic screen. Alternatively, the display where the optical pattern is displayed may be separated and distinct by the user interface 61 of the remote device 60, so that the remote device 60 allows the user to insert the setting parameter values 70 and then sends a command to a remote display to display the corresponding optical pattern 80 to be shown to the reader 40 of the medical apparatus.

Notably, the step of displaying the optical pattern on the display may alternatively be part of the remote procedure 100. In other terms, the step of displaying the optical pattern on the display may be performed at the remote distance, instead of at the acquisition distance.

When the optical pattern is used, the acquisition steps are actively performed only by the medical apparatus, which has to read the optical pattern and decode the information.

The step of emitting the setting signal may alternatively comprise emitting 201 the frequency related signal, such as the NFC, RFid or radio signal, encoding the setting parameter values 70. The remote device 60 may comprise an emitter configured to emit the frequency related signal.

The reader 40 of the medical apparatus 1 may be configured to read the optical pattern 80 encoding the setting parameter values 70: in particular the reader 40 may be configured to read, or scan, the bar-code, the QR code or the steganographic image. Alternatively, the reader 40 may be configured to read the frequency related signal, such as the NFC, RFid or radio frequency signal emitted by the remote device 60. Notably, the step of reading 203 comprises approaching the reader 40 of the medical apparatus to the remote device 60, at a distance shorter than 50 cm, in particular shorter than 20 cm, more in particular shorter than 10 cm. Optionally the step of reading comprises contacting the reader 40 of the medical apparatus to the remote device 60.

The step of acquiring 204 the setting parameter values 70 through the reading step may comprise decoding the optical pattern 80, or the frequency related signal, to obtain the setting parameter values 70. The decoding step may be performed by a control unit 50 of the medical apparatus.

The method then comprises the step of store the setting parameter values 70 in a memory of the medical apparatus to use them to set the medical treatment, such as the extracorporeal blood treatment, i.e. the dialysis treatment.

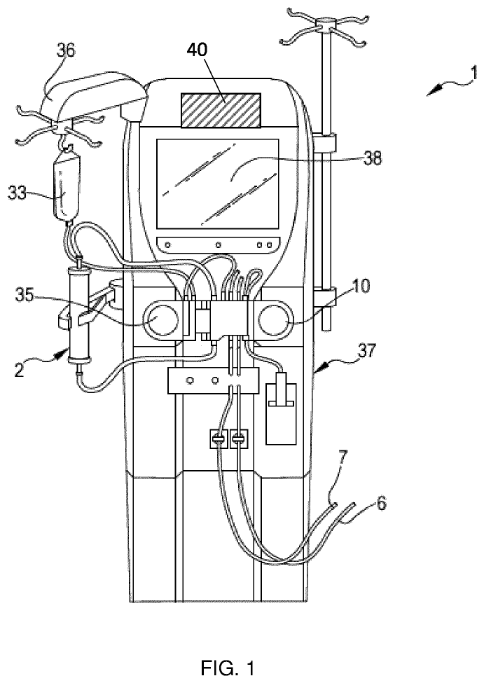

The method may also comprise steps of providing the medical apparatus with at least one disposable component necessary for the medical treatment to be carried out. For example, the disposable component may be a filter unit 2, namely the dialyzer, to be mounted on the extracorporeal blood treatment apparatus as in FIG. 1. Furthermore, the disposable component may be a bag 33 housing a fluid for infusion in the dialysis circuit or powder solution preparing a medical fluid o and/or in the blood circuit. For example, the disposable component may be a bicarbonate bag for infusion of a bicarbonate fluid solution in the patient bloodstream. Notably, the components are disposable components which need to be replaced at each treatment session. Each disposable component comprises a respective identification code, such as a label glued on the component, to identify the component type. For example, the identification code may identify whether the dialyzer is for high-flux or low-flux dialysis treatment or whether the dialyzer is directed to treat a child or an adult, or it may identify the composition of an infusion bag.

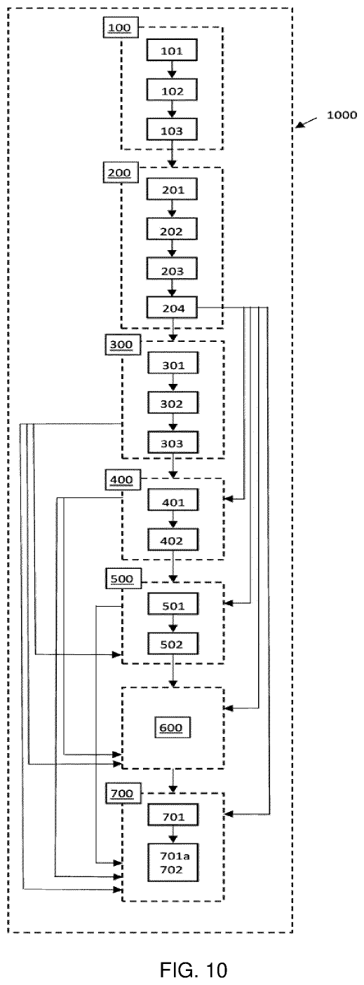

The method may further comprise a consistency procedure 300, as in FIG. 10, comprising the steps of verifying 303 consistency between the identified disposable component and the setting parameter values 70 acquired through reading the optical image from the display. As described before, the setting parameter values 70 may comprise an indication about the dialysis type, in particular whether ultrafiltration (UF), haemodialysis (HD), haemofiltration (HF), haemodiafiltration (HDF), has to be performed: the consistency procedure is directed to verify whether all the components are compatible with the dialysis type. For example, the settings may include a pre-dilution infusion flow rate and a dialysis flow rate; the dialysis type should be compatible with the imposed flow rates, namely should include a dialysis line and a pre-infusion line (being an HDF dialysis type disposable with pre-infusion). Alternatively, in case the setting parameter values 70 indicate that the patient is a child, the consistency procedure verifies whether all the components, based on the respective identification codes, are suitable for a child. Furthermore, in case the setting parameter values 70 indicates the presence of a blood warmer, the consistency procedure verifies the presence of a blood warmer associated to the dialysis machine. Obviously, the consistency procedure implies a step of reading 301 the identification code of each component: in the case wherein the identification code of the component is in the form of an optical pattern, the step of reading may be performed by the reader 40 of the medical apparatus, so that the reader 40 may be used for reading both the optical pattern of the disposable components and the optical pattern 80 encoding the setting parameter values 70. Alternatively, in the case wherein the identification code of the disposable component is in the form of a frequency related signal, the step of reading the identification code of the component may be performed by a frequency scanner distinct from the reader 40 of the apparatus.

In more detail, the consistency procedure 300 may comprise verifying 303 whether the identification code of the component matches a treatment identifier of the apparatus according to a predefined compatibility list. The treatment identifier represents the type of dialysis treatment which is going to be performed: the treatment identifier may be one of the setting parameter values. In particular the compatibility list may comprise treatment identifiers each representative of ultrafiltration (UF), haemodialysis (HD), haemofiltration (HF), haemodiafiltration (HDF), and optionally peritoneal dialysis (PD): the list further comprises a series of identification codes representatives of compatible disposable components matching the treatment identifiers. Thus, the consistency between the disposable component and the setting parameter values 70 is performed based on this predefined list which links each setting parameter value to one or more identification codes of compatible disposable components.

Then, if at least one setting parameter is not consistent with the identified disposable component for the medical apparatus, in particular of the extracorporeal blood treatment apparatus, the method includes preventing the medical apparatus to be set based on the setting parameter values 70. On the contrary, if all the setting parameter values 70 are consistent with the identified disposable component for the medical apparatus, in particular of the extracorporeal blood treatment apparatus, the method may allow the medical apparatus to be set based on the setting parameter values 70.

Furthermore, the consistency procedure 300 may comprise the step of defining 302 a respective threshold range for one or more of the setting parameter values 70 based on the identification code of the disposable component. For example, the consistency procedure may comprise the step of defining 302 a respective threshold range, based on the identification code of the component, for at least one between the patient fluid removal rate PFR for the identified patient, and two parameters for the identified patient between total weight loss WL, total treatment time TT and ultrafiltration flow rate UFR. In particular the consistency procedure may defined, based on the identification code, a threshold range for the total weight loss WL: the threshold range for the total weight loss WL may be set between 0,1 Kg and 6 Kg. Blood flow rate threshold range may be set between 50 ml/min and 600 ml/min. Of course, the proper threshold values depend upon the specific medical device, for example depends on whether the medical device is for acute patients (e.g., the blood flow range threshold would be reasonably reduced, such as between 50 ml/min and 300 ml/min) or for chronic patients (e.g., blood flow rates between 150 ml/min and 600 ml/min). Once the threshold has been set, the consistency procedure checks whether one or more setting parameter values 70 exceed the respective threshold range, namely if one or more setting parameter values 70 are out of the respective threshold range: in the case wherein one or more setting parameter values 70 exceed the respective threshold range, the method includes preventing the extracorporeal blood treatment apparatus to be set based on the setting parameter values 70.

Notably, the respective threshold range may be referred to a respective value related to at least one of the patient information 71: the method may define 302 a threshold range relative to the weight and/or the age of the patient based on the identification code of the disposable component mounted on the apparatus. For example, the identification code of the disposable component mounted on the apparatus may define a patient age threshold range between 18 and 80 years old, or between 1 and 3 years old. In the case wherein the actual age of the patient exceeds the respective age threshold range, the method prevents the extracorporeal blood treatment apparatus to be set based on said setting parameter values 70.

In other terms, the setting parameter values 70 must stay within a respective threshold range which is a function of the disposable components mounted on the medical apparatus.

As previously stated, the setting parameter values 70 may comprise patient information 71 including an identification of the patient, i.e. the name of the patient, identifying the patient which the setting parameter values 70 relate to. The method may then comprise a cross check procedure 400 performed to secure that the medical treatment is going to be performed on the right patient: in other terms, the cross check procedure 400 includes the step of associating the extracorporeal blood treatment apparatus with a patient intended to undergo the extracorporeal blood treatment. The association between the extracorporeal blood treatment apparatus and the patient is performed by providing 401 the extracorporeal blood treatment apparatus with an identification of this patient from an information source different from the optical pattern 80: for example, this identification may be provided by an identification card or an identification wearable device, such as a wearable bracelet at the wrist, of the patient which may be scanned by an identification scanner of the medical apparatus. Thus, the medical apparatus may be provided with said identification scanner configured to scan the identification card of the patient. The identification card of the patient may be a label including a bar code or a QR code, or alternatively the identification card may include an NFC or RFid circuit or a radio frequency emitter. Analogously, identification wearable device may comprise a label including a bar code or a QR code, or alternatively the identification wearable device may include an NFC or RFid circuit or a radio frequency emitter. Alternatively, the actual name of the patient may be entered into the medical apparatus manually by a user, i.e. by a nurse, through a keyboard. The identification scanner of the apparatus 1 may be the same frequency scanner of the apparatus 1 previously described and configured to scan the optical pattern of the disposable components: in this case, the frequency scanner and the identification scanner may define a single scanner configured for both identifying the patient and the components.

Furthermore, the identification scanner of the apparatus 1 may be the same reader 40 of the apparatus 1 previously described and configured to scan the optical pattern 80 encoding the setting parameter values 70: in this case, the identification scanner and the reader 40 may define a single scanner configured for both identifying the patient and reading the optical pattern 80 encoding the setting parameter values 70.

In a further embodiment, the frequency scanner, the identification scanner and the reader 40 may be combined defining a single device configured to read the optical pattern 80 encoding the setting parameter values 70, to read the identification code of the patient, and to read the identification code of the disposable components.

The cross check procedure 400 further includes the step of checking 402 the match between the patient identification of the setting parameter values 70 read through the optical pattern 80 and the identification related to the patient provided through the information source different from the optical pattern 80. If no match is found between the patient identification included in the setting parameter values 70 and the identification related to this patient provided by the information source, the cross check procedure 400 includes preventing the extracorporeal blood treatment apparatus to be set based on these setting parameter values 70. In addition, the cross check procedure 400 may also issue an alarm, i.e. an acoustic or visible alarm.

Notably, the cross check procedure 400 is performed at a cross check distance from the patient and/or from the medical apparatus 1 substantially equal to the acquisition distance. In particular, the cross check distance is very much shorter than the remote distance.

The acquisition procedure may also include a safety procedure 500 comprising a step of verifying a validity of the setting parameter values 70 acquired by the optical pattern 80 encoding the setting parameter values 70: in particular the safety procedure comprises a step of defining 501 a predefined validity range, and then verifying 502 whether all the setting parameter values 70 are within this predefined validity range. For example, the safety procedure may include verifying whether the age of the patient included in the setting parameter values 70 is within a reasonable range, i.e. between 3 and 99 years. Analogously, the safety procedure may include verifying whether the total weight loss WL included in the setting parameter values 70 is within a reasonable validity range, i.e. between 0.1 kg and 8 kg. If one or more of the setting parameter values 70 exceeds the validity range, the method prevents the extracorporeal blood treatment apparatus to be set based on these setting parameter values 70. Furthermore, if one or more of said setting parameter values 70 exceeds the validity range, the methods may also include commanding the extracorporeal blood treatment apparatus to emit an alarm signal, such as an acoustic alarm signal or an alarm displayed on a display of the extracorporeal blood treatment apparatus.

The acquisition procedure may also comprise a confirmation procedure temporally subsequent the step of acquiring the setting parameter values 70 by reading the optical pattern 80 through the reader 40. The confirmation procedure may be performed by the apparatus 1 and comprises the steps of displaying on a display, i.e. a display of the apparatus 1, one or more of the setting parameter values 70, and requiring to receive from a user an approval signal of the setting parameter values 70 received by the apparatus 1. The confirmation procedure may further includes a step of checking whether the approval signal is received by the apparatus 1.

The approval signal may comprise pushing a button, i.e. a physical button or a virtual button shown on the display of the apparatus 1: on this regard, the display of the apparatus 1 may be a touch screen.

If the approval signal is properly received by the apparatus 1, the confirmation procedure allows the step of setting the extracorporeal blood treatment apparatus 1 to be performed based on the values of said setting parameter values 70 received by the apparatus 1. On the contrary, if the approval signal is not properly received by the apparatus 1, the confirmation procedure prevents the step of setting the extracorporeal blood treatment apparatus 1 to be performed based on the values of said setting parameter values 70 received by the apparatus 1.

The requiring step of the confirmation procedure may also comprise the step of requiring one and only one approval signal representative of approval of all the setting parameter values 70 received by the apparatus 1.

Alternatively, the requiring step of the confirmation procedure may comprise a step of partitioning the setting parameter values 70 into two or more groups each one comprising respective setting parameter values 70 received by the apparatus 1, and requiring an approval signal for each group of setting parameter values 70.

The setting parameter values 70 may be partitioned in these groups based on a category of each setting parameter value: i.e. at least a flow rate group may be provided comprising only the setting parameter values related to a flow rate to set the extracorporeal blood treatment apparatus 1. For example, an identification group may be provided comprising only the setting parameter values related to identification of the patient.

Furthermore, a therapy group may be provided comprising only the setting parameter values related to the therapy configuration 72. Notably, the confirmation procedure may comprise providing at least one between the flow rate group, the identification group and the therapy group or all the flow rate, identification and therapy groups.

In alternative, the requiring step of the confirmation procedure may comprise requiring, for each setting parameter value 70 of the setting parameter values 70, a respective approval signal representative of approval of each setting parameter value 70 received by the apparatus 1.

The present method may also comprise a step of pairing 600 the remote device 60 with the extracorporeal blood treatment apparatus based on an authentication code to allow an authorized user to perform the pairing step: the authentication code may comprise at least one between a password, a serial number, a fingerprint, a facial recognition algorithm. The pairing may be based on NFC or Bluetooth protocols: for example the remote device 60, i.e. a tablet or a smartphone, is paired to the medical apparatus through a Bluetooth connection.

If the pairing step 600 is successful, the method performs the acquisition procedure 200: on the contrary, if the pairing step is not successful, the method prevents the acquisition procedure 200 to be carried on, or the method prevents the setting parameter values 70 to set the extracorporeal blood treatment apparatus.

Notably, the method 1000 may include pairing 600 the remote device 60 with the extracorporeal blood treatment apparatus 1 every time the setting parameter values 70 are acquired and read by the reader 40. Optionally, the method 1000 may include pairing 600 the remote device 60 before reading the optical pattern 80 by the reader 40 of the medical apparatus: once the remote device 60 is paired to the medical apparatus, the method 1000 allows the reading step 203 and the acquisition procedure to be performed.

In an embodiment, the optical pattern 80 which encodes the setting parameter values 70 may further include the authentication code to allow pairing of the remote device 60 with the medical apparatus, in particular with the extracorporeal blood treatment apparatus. For example, the barcode or the QR code may encode both the setting parameter values 70 and the authentication code to perform the pairing step.

According to an optional embodiment, the step of displaying 201 the optical pattern 80 on the display of the remote device 60 is subordinated to a successful pairing between the remote device 60 and the medical apparatus: if the paring is not successful, the method prevents the optical pattern 80 to be displayed on the display 62 of the remote device 60. Notably, in this optional embodiment, the remote device 60 comprises both the display 62 and the input device 63 (i.e. as a smartphone or a tablet).

In a further embodiment that does not require any pairing between the remote device and the medical device, the operator has to enable the optical code reading at the medical device before the acquisition procedure. For example, the operator has to input his/her ID directly through the medical device user interface and exclusively upon verification by the medical device of the proper authorization to set the medical device through the optical code, the latter may be acquired and/or used by the device. This avoids the need of any pairing or by-directional communication between the remote device and the medical device.

In an embodiment wherein the user interface 61 of the remote device 60 comprises both the display 62 and a input device 63 for entering the setting parameter values 70, the method further comprises a local procedure 700, temporally interposed between the remote procedure and the acquisition procedure, comprising the steps of entering 701 one or more further setting parameter values 70 into the remote device 60 through the user interface 61 of the remote device 60. The further setting parameter values 70 comprise one or more of the setting parameter values 70, for example at least one between a patient prescription 73, a therapy configuration 72 and a patient information 71. Alternatively or in addition, the local procedure comprises the step of modifying 701a or updating 702, through the user interface 61 of the remote device 60, one or more of the setting parameter values 70 entered during the remote procedure. The user may update setting parameter values related to the prescription or the patient information 71, such as the weight or the hematocrit value. Notably, the local procedure 700 is performed by a user at a local distance from the patient and/or from the medical apparatus, while the remote procedure 100 is performed by a user at a remote distance from the patient and/or from the medical apparatus: the local distance is shorter than the remote distance. In particular the local distance is shorter than 10 meters, in particular shorter than 5 meters, more in particular shorter than 3 meters, and more in particular shorter than 1 meter. De facto, the local distance corresponds to the acquisition distance of the acquisition procedure: in other terms, the local procedure 700 and the acquisition procedure 200 are performed substantially at the same place, in particular at the same distance with respect to the patient and/or to the medical apparatus.

FIG. 9 shows a flow chart according to an embodiment of the present invention comprising the main steps of the remote procedure 100 and the acquisition procedure 200.

FIG. 10 shows a flow chart according to a further embodiment of the present invention comprising the main steps of the remote procedure 100, the acquisition procedure 200, the consistency procedure 300, the cross check procedure 400, the safety procedure 500, the pairing step 600, and the local procedure 700. In particular, the flow chart of FIG. 10 does not limit the method to include all these steps 100, 200, 300, 400, 500, 600 and 700: the method may indeed comprise, in addition to the remote procedure 100 and the acquisition procedure 200, one or more between the consistency procedure 300, the cross check procedure 400, the safety procedure 500, the pairing step 600, and the local procedure 700 in any combination thereof as pointed out by the side flow arrows in FIG. 10.

### System

The present invention is also directed to a system 90 comprising both the medical apparatus 1 and the remote device 60.

The remote device 60 may be a portable device, such as a smartphone, a tablet, or the like as in FIGS. 5-8. The remote device 60 comprises at least a user interface 61 configured to allow a user to enter the setting parameter values 70, and a controller 51 to allow a user to enter the setting parameter values 70 through the user interface 61. In particular the user interface 61 comprises an input device 63 such as a keyboard.

Furthermore, the controller 51 is also configured to generate a setting signal encoding the setting parameter values 70: the setting signal may be an optical pattern 80 or a frequency related signal, such as an NFC, RFid or radio frequency signal encoding the setting parameter values 70.

The user interface 61 of the remote device 60 may also comprise a display 62, connected to the controller 51, and configured to visualize, namely to display on screen, the optical pattern 80. On this regard, the controller 51 is configured to encode the setting parameter values in the optical pattern 80, and then to display this optical pattern 80 on the display 62 of the remote device 60 or on a remote display (not shown in the attached figures).

Analogously, the controller 51 may be configured to encode the setting parameter values in the optical pattern 80, and then to emit the frequency related signal through the emitter.

Furthermore, the remote device 60 comprises an electric battery configured to supply the controller 51 and to the user interface 61, namely the display 62 and the input device 63.

Alternatively, the remote device 60 comprises an emitter configured to emit the frequency related signal, such as an NFC, RFid or radio frequency signal encoding the setting parameter values 70. In this case, the remote device 60 may not comprise the display.

According to an embodiment, the remote device 60 defines a single body, as in FIGS. 5-8, comprising a case carrying at least the display 62, the battery, the input device 63, and the controller 51. The display 62 and the input device 63 may be combined defining a touchscreen.

Alternatively, the remote device 60 defines a single body comprising a case carrying at least the emitter, the battery, the input device 63, and the controller 51.

The controller 51 of the remote device 60 is configured to allow to perform the remote procedure 100, such as the steps 101, 102 and 103 previously described: in particular the controller 51 is configured to allow entry of values for setting parameter values 70 via the user interface 61. The setting parameter values 70, which have been already described before in the method description, define a setting of the extracorporeal blood treatment apparatus and are related to an identified patient. The setting parameter values 70 are part of a patient prescription 73 for the identified patient, and comprise at least one of a patient fluid removal rate PFR for the identified patient, and two parameters for the identified patient between total weight loss, total treatment time, and ultrafiltration flow rate.

The previous description related to the method often refers generally to a medical apparatus 1 for conciseness: anyhow, the medical apparatus may be the extracorporeal blood treatment apparatus, such as a dialysis apparatus as shown in FIG. 1. The dialysis apparatus may be configure to run an ultrafiltration treatment UF, a haemodialysis treatment HD, a haemofiltration treatment HF, or a haemodiafiltration treatment HDF. The dialysis apparatus 1 may be directed to chronic or acute treatments. Furthermore, the extracorporeal blood treatment apparatus may be an extracorporeal membrane oxygenation apparatus configured to remove CO₂ and/or oxygenate patient blood. In one embodiment, the extracorporeal blood treatment apparatus may be configured to perform a dialysis treatment and to remove CO₂ with a membrane oxygenator (in other terms, the apparatus includes one filtration unit for the dialysis treatment and a membrane oxygenator to remove CO₂. Alternatively, the medical apparatus 1 may be a peritoneal dialysis apparatus or an infusion pump.

The medical apparatus of the present invention comprises a control unit 50, schematically shown in FIG. 1, configured to perform the acquisition procedure 200 previously described in the method section. In more detail the medical apparatus comprises a reader 40: the control unit 50 is configured to command the reader 40 to acquire the setting parameter values 70 by reading the optical pattern 80, and then setting the medical apparatus based on the values of the setting parameter values 70. In other terms, the control unit 50 is configured to perform the reading step 203 previously described.

The medical apparatus 1 may also have a memory, operatively connected to the control unit 50, configured to store the setting parameter values 70.

The control unit 50 is also configured to perform the consistency procedure 300: in particular the control unit 50 of the medical apparatus 1 is configured to read 301 the identification code of each component, and verify 303 consistency between the identified disposable component and the setting parameter values 70 acquired through reading the optical image from the display 62 of the remote device 60. Furthermore, the control unit 50 may also be configured to perform the step, within the consistency procedure, of defining 302 the respective threshold range, based on the identification code of the component, for at least one between the patient fluid removal rate PFR for the identified patient, and two parameters for the identified patient between total weight loss WL, total treatment time TT and ultrafiltration flow rate UFR. More in general, the control unit 50 of the medical apparatus 1 may be configured to define 302 the respective threshold range for one or more of the setting parameter values 70 based on the identification code of the disposable component.

The control unit 50 may also be configured to perform the cross check procedure 400 performed to secure that the medical treatment is going to be performed on the right patient: in other terms, the control unit 50 may be configured to allow associating the extracorporeal blood treatment apparatus with a patient intended to undergo the extracorporeal blood treatment.

In particular, the control unit 50 may be configured for receiving 401 an identification of this patient from an information source different from the optical pattern 80: then the control unit 50 is configured for checking 402 the match between the patient identification of the setting parameter values 70 read through the optical pattern 80 and the identification related to the patient provided through the information source different from the optical pattern 80. If no match is found between the patient identification included in the setting parameter values 70 and the identification related to this patient provided by the information source, the control unit 50 is configured to prevent the extracorporeal blood treatment apparatus to be set based on these setting parameter values 70. The control unit 50 may also be configured to perform the safety procedure 500 to check a validity of the setting parameter values 70, i.e. acquired by the optical pattern 80 encoding the setting parameter values 70. The control unit 50 is configured for defining 501 the predefined validity range, and then verifying 502 whether all the setting parameter values 70 are within this predefined validity range, as described in detail in the method section.

The control unit 50 of the medical apparatus 1 and the controller 51 of the remote device 60 may also be configured to perform the step of pairing 600 the remote device 60 with the extracorporeal blood treatment apparatus 1 based on an authentication code. In particular this pairing step may be performed by both the controller 51 and the control unit 50: the pairing step may be performed by using NFC or Bluetooth protocols. The controller 51 of the remote device 60 may also be configured to perform the local procedure 700 temporally interposed between the remote procedure and the acquisition procedure: the controller 51 may be configured to allow a user for entering 701 one or more further setting parameter values 70 into the remote device 60 through the user interface 61 of the remote device 60. In other terms, the controller 51 is configured to receive, from the user interface, further setting parameter values after the remote procedure. The further setting parameter values 70 comprise one or more of the setting parameter values 70, for example at least one between a patient prescription 73, a therapy configuration 72 and a patient information 71.

Alternatively or in addition, the controller 51 of the remote device 60 may be configured to allow a user for modifying 701a or updating 702, through the user interface 61 of the remote device 60, one or more of the setting parameter values 70 entered during the remote procedure. Notably, the controller 51 is configured to perform the local procedure 700 at a local distance from the patient substantially equal to the acquisition distance kept during the acquisition procedure. In particular the local distance is much shorter than the remote distance.

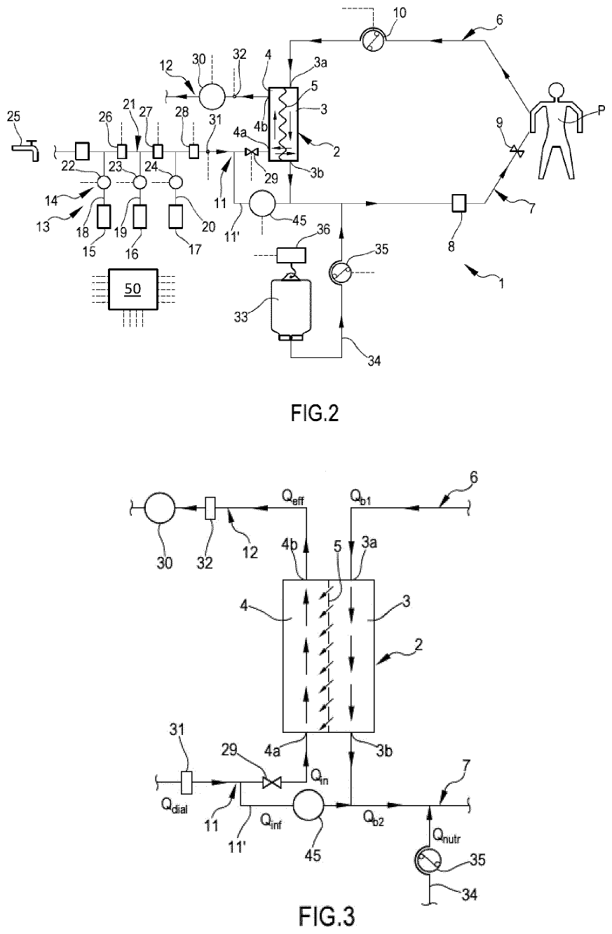

According to an embodiment shown in FIGS. 2 and 3, the extracorporeal blood treatment apparatus comprises at least a filtration unit 2 having a primary chamber 3 and a secondary chamber 4 separated by a semi-permeable membrane 5. The extracorporeal blood treatment apparatus 1 further comprises a blood circuit comprising a blood withdrawal line 6, extending between a first end connected to an inlet of the primary chamber and a second end for connection to the patient P. The extracorporeal blood treatment apparatus further comprises a blood return line 7 extending between a first end connected to an outlet of the primary chamber 3 and a second end for connection to the patient P. In addition, the extracorporeal blood treatment apparatus comprises a blood pump 10 to circulate blood in the blood circuit, and an effluent line 12 connected to an outlet of the secondary chamber 4. The control unit 50 is also configured to control at least the blood pump 10, for example to activate or vary the speed of the blood pump 10.

Here after, a description of a Chronic (long-term) hemodialysis (HD) apparatus, compatible with the setting method and the system of the present invention, is provided in detail. Notably, the description of the following chronic dialysis circuit is disclosed as an exemplary embodiment, without limiting the present invention to chronic hemodialysis. Indeed, the present setting method may also be performed in case of acute extracorporeal blood treatments.

### Extracorporeal Blood Treatment Apparatus 1

Chronic (long-term) hemodialysis (HD) therapy systems used to treat patients with chronic kidney failure. HD therapy is usually performed several times a week (e.g. 3 times a week) and each treatment has an average duration of some hours (e.g. 3.5-4.5 hours).

An apparatus 1 for extracorporeal blood treatment for chronic (long term) therapy is represented in FIGS. 1 and 2. The apparatus 1 comprises a filtration unit 2 having a primary chamber 3 and a secondary chamber 4 separated by a semi-permeable membrane 5. Depending upon the treatment, the semi-permeable membrane 5 of the filtration unit 2 may be selected to have different properties and performances.

A blood circuit is coupled to the primary chamber 3 of the filtration unit 2. The blood circuit comprises a blood withdrawal line 6 connected to an inlet 3a of the primary chamber 3, a blood return line 7 connected to an outlet 3b of the primary chamber 3. The withdrawal line 6 and blood return line 7 are configured for connection to a cardiovascular system of a patient “P”. During the therapy, the patient has two accesses (arterial/vein) where blood flows in/out from the body, wherein the average blood flow is usually between 250 and 500 ml/min.

In use, the blood withdrawal line 6 and the blood return line 7 are connected to a needle or to a catheter or other access device which is then placed in fluid communication with the patient “P” vascular system, such that blood may be withdrawn through the blood withdrawal line 6, flown through the primary chamber 3 and then returned to the patient's vascular system through the blood return line 7. An air separator, such as a de-aeration chamber 8, may be present on the blood return line 7. Moreover, a monitor valve 9 may be present on the blood return line 7, downstream the de-aeration chamber 8.

The blood flow through the blood circuit is controlled by a blood pump 10, for instance a peristaltic blood pump, acting either on the blood withdrawal line 6 or on the blood return line 7. The embodiment of FIG. 2 shows the blood pump 10 coupled to a pump section of the withdrawal line 6. Alternatively, a blood pump may be provided both on the blood withdrawal line 6 and on the blood return line 7. A control unit 50 is connected and controls the blood pump 10 to regulate a blood flow rate.

A dialysis circuit is connected to the secondary chamber 4 of the filtration unit 2 and comprises a dialysis supply line 11 connected to an inlet 4a of the secondary chamber 4 and a dialysis effluent line 12 connected to an outlet 4b of the secondary chamber 4 and to a drain, not shown. The dialysis effluent line discharges a spent dialysis fluid into the drain.

The dialysis supply line 11 is connected to a preparation device 13 for preparing a fresh dialysis fluid. The preparation device 13 comprises a regulating device 14 for regulating the composition of the fresh dialysis fluid.

In the example of FIG. 2, the preparation device 13 comprises three containers of concentrate 15, 16, 17 located on respective injection lines 18, 19, 20 which are predisposed to supply substances such as electrolytes, buffer agents or others towards a preparation line 21 of the fresh dialysis fluid. The concentrate containers 15, 16, 17 may comprise concentrates in the liquid state or solid state, for example powder.

The regulating device 14 comprises injection pumps 22, 23, 24 placed on the injection lines 18, 19, 20 to move the fluid along the respective injection line 18, 19, 20 towards the preparation line 21 which collects the liquid, for example water, from a source 25. The preparation line 21 is located upstream the dialysis supply line 11 and has one end connected to the source 25, e.g. a deionized/purified water source or a reverse osmosis water plant, and an opposite end connected to the dialysis supply line 11. The source 25 may comprise the water source, as shown, or a source of ultra-pure liquid.

Concentration or conductivity sensors 26, 27, 28 are located on the preparation line 21 and are able to provide the control unit 50 with a signal related to conductivity or concentration of a predetermined substance (for example sodium) of the fluid crossing the preparation line 21 such that the control unit 50 is able to control the injection pumps 22, 23, 24 in order to regulate the conductivity Cd or concentration, for example of sodium [Na], of the liquid crossing the dialysis supply line 11. A fluid check organ 29 may be used to selectively enable or inhibit a passage of fluid across the dialysis line 21 and into the filtration unit 2 in case the liquid does not meet the required parameters.

An infusion line 11′ departs from the dialysis supply line 11, in particular downstream the preparation device 13, and is connected to the blood return line 7 to infuse part of the fresh dialysis fluid into the blood circuit. An auxiliary infusion pump 45 may be coupled to the infusion line 11′ to deliver said part of the fresh dialysis fluid into the blood circuit. The infusion line 11′ may alternatively or in addition being connected to the blood withdrawal line 6 (in particular downstream the blood pump 10) for pre-infusing a substitution fluid.

An ultrafiltration device is configured to achieve a fluid removal from the patient through the semi-permeable membrane 5 of the filtration unit 2. The ultrafiltration device comprises a dialysis pump 30 located on the dialysis effluent line 12. In a variant embodiment, a first dialysis pump is coupled to the dialysis supply line 11 and a second dialysis pump coupled to the dialysis effluent line 12. A first flow-meter 31 is active on the dialysis supply line 11 and is placed between the fluid check organ 29 and the inlet 4a of the secondary chamber 4. A second flow-meter 32 is active on the dialysis effluent line 12 and is placed between the outlet 4a of the secondary chamber 4 and the dialysis pump 30.

The infusion line 11′ is connected to the dialysis supply line 11 between the first flow-meter 31 and the inlet 4a of the secondary chamber 4. The first flow-meter 31 and the second flow-meter 32 are connected to the control unit 50 and are configured to determine an ultrafiltration rate UFR.

The ultrafiltration rate UFR is a difference between the spent dialysis fluid exiting the outlet 4b of the secondary chamber 4 and the prepared fresh dialysis fluid routed to the inlet 4a of the secondary chamber 4 and the fluid optionally infused into the blood circuit through the infusion line 11′ (FIG. 3).

As shown in FIG. 3, Q_{b1} is the blood flow rate entering the primary chamber 3 through the inlet 3a, Q_{b2} is the blood flow rate leaving the primary chamber 3 through the outlet 3b, Qᵢₙ is the flow rate entering the secondary chamber 4 through the inlet 4a, Q_{inf} is the flow rate crossing the infusion line 11′, wherein:

$${Q}_{dial} = {Q}_{i n} + {Q}_{inf}$$

The first flow-meter 31 and the second flow-meter 32 provide the control unit 50 with an instant value of the respective flows and thus enable the control unit 50 to calculate an instant ultrafiltration rate UFR. Alternatively, a differential sensor may be provided, active on the dialysis supply line 11 and on the dialysis effluent line 12 and therefore able directly to provide a signal relating to the ultrafiltration rate UFR. Instead of flowmeters, balance chambers may operatively be coupled to the dialysis circuit is provided. The balance chambers principle operates so that the amount of fluid entering into the first chamber on the dialysis supply line 11 is equal to the amount of fluid exiting from the dialysis effluent line 12.

To achieve the ultrafiltration, an ultrafiltration line (not represented) is added to the effluent line upstream the balancing chamber. An ultrafiltration pump removes the desired amount of ultrafiltered liquid before that the spent dialysis liquid reaches the second balance chamber thereby achieving an ultrafiltered volume. Of course absolute volume variation through ultrafiltration line may be measured too.

The apparatus 1 may further comprise one infusion bag 33 housing an infusion solution such replacement fluid or a nutritional substance, and an infusion line 34 having a first end in fluid communication with the infusion bag 33 and a second end connected to the blood return line 7 for infusing the solution into the patient vascular system through said blood return line 7. In an alternative embodiment, the infusion solution may be infused directly into the patient vascular system. An infusion pump 35 is coupled to the infusion line 34 to deliver the infusion solution through the infusion line 34. A sensing element in the form of a weighing device 36 is configured to weigh the infusion bag 33 while the infusion solution is infused and to provide afirst signal allowing calculation of a first parameter W related to a weight of the infusion bag 33 and, therefore, to the weight or volume of infusion solution contained in the infusion bag 33. In general, the weighing device 36 provides the time variation of the bag weight which is directly linked to the actual flow rate of the infusion solution through the infusion line.

Indeed, the control unit knows the infusion line cross section and may therefore easily and very accurately calculate the actual flow rate of the infusion solution through the infusion line starting from measurements of weight/weight variation over time of the corresponding bag. Optionally the first parameter is the weight of the infusion bag 33 measured over time. As an alternative, a flow meter on the infusion line may be used as a sensing element.

Finally, though less precise, the pump speed may be used to determine flow rate through the infusion line, the speed being monitored through a suitable sensor, such as a Hall sensor or a sensor sensitive to pump electric parameters such as resistance or power consumption.

As shown in FIG. 1, the apparatus 1 may comprise a main body 37 provided with a base resting on the ground and hosting all the components of said apparatus 1, i.e.: the filtration unit 2, the blood circuit, the blood pump 10, the dialysis circuit, the preparation device 13, the weighing device 36, the infusion line 34, the infusion pump 35, the control unit 50. The weighing device 36 is a weight scale and is configured to hang the infusion bag 33. The control unit 50 is housed in the main body 37. The infusion pump 35 and the blood pump 10 are peristaltic pumps supported by the main body 37. Each peristaltic pump comprises an actuator or motor, not shown, connected to a rotor. The rotor of the infusion pump 35 and the rotor of the blood pump 10 are placed on a front face of the main body 37.

The blood circuit and the filtration unit 2 are disposable (i.e. they are disposed after each blood treatment) and are coupled in removable manner to the main body 37. The pump section of the withdrawal line 6 is coupled in removable manner to the rotor of the blood pump 10. The infusion line 34 is disposable and is coupled in removable manner to the main body 37. A pump section of the infusion line 34 is coupled in removable manner to the infusion pump 35.

The dialysis circuit is not-disposable and is configured to be sterilized after each blood treatment. Tubes, pumps, sensors of the dialysate circuit are not configured to be replaced after each treatment but only for maintenance purposes or in case of faults. Therefore, the dialysis circuit is integrated in the main body 36 and mounted fixed in or on the main body 37. The dialysis pump 30 is a volumetric pump crossed by the effluent fluid and is mounted in the main body 37.

The control unit 50 is contained in or supported by the main body 37. The control unit 50 may be connected to the blood pump 10, the weighing device 36, the infusion pump 35, the injection pumps 22, 23, 24 of the regulating device 14, the concentration or conductivity sensors 26, 27, 28, the first flow-meter 31, the second flow-meter 32, the fluid check organ 29 and to the dialysis pump 30 of the ultrafiltration device. The control unit 50 controls the weighing device to measure the weight at instants of time, e.g. with an acquisition frequency 1 Hertz. The control unit 50 is also connected to a display screen 38 (FIG. 1) mounted on the main body 37.

The control unit 50 may comprise a digital processor (CPU) with memory (or memories), an analogical type circuit, or a combination of one or more digital processing units with one or more analogical processing circuits.

In the present description and in the claims it is indicated that the control unit 50 is “configured” or “programmed” to execute steps: this may be achieved in practice by any means which allow configuring or programming the control unit 50. For instance, in case of a control unit 50 comprising one or more CPUs, one or more programs are stored in an appropriate memory: the program or programs containing instructions which, when executed by the control unit 50, cause the control unit 50 to execute the steps described and/or claimed in connection with the control unit 50. Alternatively, if the control unit 50 is of an analogical type, then the circuitry of the control unit 50 is designed to include circuitry configured, in use, to process electric signals such as to execute the control unit 50 steps herein disclosed.

The control unit 50 is configured to or programmed for receiving signals from the sensors and from other inputs and for commanding pumps and valves according to said signals, in order to perform the blood treatment and to administer infusion products to the patient during blood treatment. The control unit 50 is also configured to or programmed for displaying data on the display screen 38.

1-19. (canceled)

20. A system comprising an extracorporeal blood treatment apparatus and a remote device, the extracorporeal blood treatment apparatus comprising:
a filtration unit having a primary chamber and a secondary chamber separated by a semi-permeable membrane;
a blood circuit comprising:
a blood withdrawal line extending between a first end connected to an inlet of the primary chamber and a second end for connection to a patient; and
a blood return line extending between a first end connected to an outlet of the primary chamber and a second end for connection to the patient;
a blood pump to circulate blood in the blood circuit;
an effluent line connected to an outlet of the secondary chamber;
a reader configured to read optical patterns;
a control unit configured to control the blood pump and connected to the reader;
wherein the remote device comprises a user interface and a controller configured to perform a remote procedure comprising:
allowing entry of values for setting parameter values via the user interface, wherein the setting parameter values define a setting of the extracorporeal blood treatment apparatus and are related to an identified patient, the setting parameter values comprising part of a patient prescription for the identified patient, the setting parameter values comprising at least one of:
a patient fluid removal rate for the identified patient; and
two parameters for the identified patient selected from total weight loss, total treatment time, and ultrafiltration flow rate;
generating an optical pattern encoding the setting parameter values, the optical pattern being variable as a function of the entered setting parameter values;
and wherein the control unit of the extracorporeal blood treatment apparatus is configured to perform an acquisition procedure comprising:
acquiring the setting parameter values by reading the optical pattern through the reader; and
setting the extracorporeal blood treatment apparatus based on the values of the setting parameter values.

21. The system of claim 20, wherein the total weight loss parameter is a function of both the total treatment time and the ultrafiltration flow rate.

22. The system of claim 20, wherein the total weight loss parameter is a function of both the total treatment time and the ultrafiltration flow rate according to the following relationship:
$$W L = U F R \cdot T T$$
wherein:
WL=total weight loss;
UFR=ultrafiltration flow rate; and
TT=total treatment time.

23. The system of claim 20, wherein the optical pattern comprises an image encoding information comprising the setting parameter values.

24. The system of claim 20, wherein the remote device comprises a portable device defining one body and physically comprising both an input device configured to allow a user to enter the setting parameter values, and a display configured to visualize the optical pattern, wherein the controller of the remote device is configured to visualize the optical pattern on the display.

25. The system of claim 20, wherein the setting parameter values comprise a plurality of instructions for the extracorporeal blood treatment apparatus to subsequently perform the extracorporeal blood treatment treatment on the patient, and wherein the setting parameter values comprise patient information including a patient identification and at least one information selected from a patient age, a patient weight, a patient haematocrit value, a patient gender, and a patient related disease.

26. The system of claim 20, wherein the setting parameter values comprise a therapy configuration including one or more information selected from:
a dialysis treatment type, comprising one of an ultrafiltration treatment, a haemodialysis treatment, a haemofiltration treatment, and a haemodiafiltration treatment;
a disposable fluid line set type representative of the dialysis treatment type;
a dialysis filter or filter-set type representing a dialyzer type or a disposable set including the dialyzer type;
an anticoagulation setting for the extracorporeal blood treatment apparatus, the anticoagulation setting being selected from heparin anticoagulation and regional anticoagulation; and
presence or absence of a blood warmer.

27. The system of claim 20, wherein the patient prescription comprises one or more flow rate values to be set to the extracorporeal blood treatment apparatus, the one or more flow rate values being selected between a blood flow rate, a Pre Blood Pump flow rate, a syringe flow rate, a fresh dialysis flow rate, and a replacement fluid flow rate.

28. The system of claim 20, further comprising:
a disposable component configured for coupling to the extracorporeal blood treatment apparatus for a treatment to be carried out, the disposable component comprising an identification code to identify the disposable component;
wherein the control unit of the extracorporeal blood treatment apparatus is configured to perform a consistency procedure comprising:
receive the identification code of the disposable component to identify the disposable component, the control unit configured to receive the identification code from:
the reader of the extracorporeal blood treatment apparatus, wherein the identification code of the disposable component comprises a respective optical pattern; or
a frequency scanner, distinct from the reader of the apparatus, wherein the identification code of the disposable component comprises a frequency related signal;
verifying consistency between the identified disposable component and the setting parameter values acquired through reading the optical image from the display;
when one or more of the setting parameter values are not consistent with the identified disposable component for the extracorporeal blood treatment apparatus, prevent the extracorporeal blood treatment apparatus to be set based on the setting parameter values.

29. The system of claim 28, further comprising a plurality of disposable components configured for coupling to the extracorporeal blood treatment apparatus for a treatment to be carried out, wherein each disposable component comprises a respective component identifier encoding a respective identification code,
wherein the setting parameter values comprise a treatment identifier representative of the dialysis treatment to be set in the extracorporeal blood treatment apparatus, the dialysis treatment selected from an ultrafiltration treatment, a haemodialysis treatment, a haemofiltration treatment, and a haemodiafiltration treatment;
wherein the consistency procedure comprises verifying whether the respective component identifier of the disposable component matches the treatment identifier of the extracorporeal blood treatment apparatus according to a predefined compatibility list,
wherein the predefined compatibility list comprises, for each dialysis treatment selected from an ultrafiltration treatment, a haemodialysis treatment, a haemofiltration treatment, and a haemodiafiltration treatment, a series of identification codes representative of disposable components compatible with the respective dialysis treatment.

30. The system of claim 20, wherein the setting parameter values comprise patient information including an identification of the patient identifying the patient to which the setting parameter values relate,
wherein the control unit of the extracorporeal blood treatment apparatus is configured to perform a cross check procedure including:
associating the extracorporeal blood treatment apparatus with a patient intended to undergo the extracorporeal blood treatment;
providing the extracorporeal blood treatment apparatus with an identification of the patient from an information source different from the optical pattern;
checking for a match between the patient identification of the setting parameter values read through the optical pattern and the identification of the patient provided through the information source different from the optical pattern;
when no match is found between the patient identification of the setting parameter values and the identification of the patient from the information source, preventing the extracorporeal blood treatment apparatus to be set based on the setting parameter values and issuing an alarm.

31. The system of claim 20, wherein the acquisition procedure comprises a safety procedure comprising verifying a validity of the setting parameter values acquired by the optical pattern, the safety procedure comprising verifying whether all the setting parameter values are within a predefined validity range,
when one or more of the setting parameter values exceeds the predefined validity range, preventing the extracorporeal blood treatment apparatus to be set based on the setting parameter values,
and wherein when one or more of the setting parameter values exceeds the predefined validity range, the safety procedure comprises commanding the extracorporeal blood treatment apparatus to emit an alarm signal.

32. The system of claim 20, wherein the control unit of the apparatus and the controller of the remote device are configured to pair the remote device with the extracorporeal blood treatment apparatus based on an authentication code to allow an authorized user to perform the pairing so that:
when the pairing is successful, the acquisition procedure is performed;
when the pairing is not successful, the acquisition procedure is prevented from being carried on, or the setting parameter values are prevented from setting the extracorporeal blood treatment apparatus.

33. The system of claim 32, wherein the pairing is based on a physical proximity wireless protocol and the authentication code comprises at least one of a password, a serial number, a fingerprint, and a facial recognition algorithm.

34. The system of claim 20, wherein the remote procedure is performed at a remote distance from the patient and from the extracorporeal blood treatment apparatus, and the acquisition procedure is performed at an acquisition distance from the patient and from the medical apparatus, wherein the remote distance is longer than the acquisition distance,
wherein the remote procedure is performed:
remotely at a safety distance with respect to the patient and with respect to the medical apparatus, wherein the safety distance is higher than 20 meters; and/or
separately from the patient by a barrier,
wherein the barrier and/or the safety distance being configured to isolate a user, in charge of entering the setting parameter values into the remote device, from the patient to avoid bacteria and/or virus transmission between the patient and the user,
and wherein the acquisition procedure is performed close to the extracorporeal blood treatment apparatus, and close to the patient, wherein during the acquisition procedure the user, in charge of performing the acquisition procedure, is at a distance from the patient shorter than 3 meters.

35. The system of claim 34, wherein the control unit of the extracorporeal blood treatment apparatus is configured to perform a cross check procedure including:
associating the extracorporeal blood treatment apparatus with a patient intended to undergo the extracorporeal blood treatment;
providing the extracorporeal blood treatment apparatus with an identification of the patient from an information source different from the optical pattern;
checking for a match between the patient identification of the setting parameter values read through the optical pattern and the identification of the patient provided through the information source different from the optical pattern;
when no match is found between the patient identification of the setting parameter values and the identification of the patient from the information source, preventing the extracorporeal blood treatment apparatus to be set based on the setting parameter values and issuing an alarm.
wherein the cross check procedure is performed at a cross check distance substantially equal to the acquisition distance, wherein the setting parameter values comprise patient information including an identification of the patient identifying the patient to which the setting parameter values relate,.

36. The system of claim 20, wherein the user interface of the remote device comprises both the display and a input device for entering the setting parameter values,
wherein the controller of the remote device is configured to perform a local procedure temporally interposed between the remote procedure and the acquisition procedure, the local procedure comprising:
entering or allowing a user to enter one or more further setting parameter values into the remote device through the user interface of the remote device, the further setting parameter values comprising one or more of a patient prescription, a therapy configuration, and a patient information; and/or
modifying, updating, or allowing a user to modify or update, through the user interface of the remote device, one or more of the setting parameter values entered during the remote procedure,
wherein the local procedure is performed by a user at a local distance from the patient and from the extracorporeal blood treatment apparatus, while the remote procedure is performed by a user at a remote distance from the patient and from the extracorporeal blood treatment apparatus, wherein the local distance is shorter than the remote distance,
and wherein the local procedure and the acquisition procedure are performed substantially at the same place and at the same distance with respect to the patient and to the extracorporeal blood treatment apparatus.

37. The system of claim 20, wherein the acquisition procedure comprises a confirmation procedure temporally subsequent to acquiring the setting parameter values by reading the optical pattern through the reader,
wherein the confirmation procedure comprises:
displaying on a display of the apparatus one or more of the setting parameter values;
requiring, from a user, an approval signal of the one or more of the setting parameter values received by the apparatus;
checking whether the approval signal is received;
when the approval signal is received by the apparatus, allowing setting of the extracorporeal blood treatment apparatus based on the values of the setting parameter values received by the apparatus;
when the approval signal is not received by the apparatus, preventing setting of the extracorporeal blood treatment apparatus based on the values of the setting parameter values received by the apparatus.

38. The system of claim 37, wherein requiring an approval signal from a user during the confirmation procedure comprises:
requiring one and only one approval signal representative of approval of all the setting parameter values received by the apparatus; or
partitioning the setting parameter values into two or more groups, each group comprising respective setting parameter values received by the apparatus, and requiring an approval signal for each group of setting parameter values.

39. The system of claim 38, wherein the setting parameter values are partitioned in the groups based on a category of each setting parameter value, wherein at least a flow rate group is provided comprising only setting parameter values related to flow rates to set the extracorporeal blood treatment apparatus.

40. The system of claim 20, wherein a variation of the setting parameter values entered corresponds to a variation in the optical pattern displayed on the user interface of the remote device, the optical pattern being displayed on the display of the remote device, and wherein displaying the optical pattern on the display is performed during the acquisition procedure and temporally after the remote procedure, and is subordinated to a successful pairing between the remote device and the extracorporeal blood treatment apparatus, wherein the optical pattern in not displayed when the paring is not successful.

41. A system comprising an extracorporeal blood treatment apparatus, a remote device and a disposable component to be coupled to the extracorporeal blood treatment apparatus for a treatment to be carried out, the extracorporeal blood treatment apparatus comprising:
a filtration unit having a primary chamber and a secondary chamber separated by a semi-permeable membrane;
a blood circuit comprising:
a blood withdrawal line extending between a first end connected to an inlet of the primary chamber and a second end for connection to a patient; and
a blood return line extending between a first end connected to an outlet of the primary chamber and a second end for connection to the patient;
a blood pump to circulate blood in the blood circuit;
an effluent line connected to an outlet of the secondary chamber;
a reader configured to read optical patterns;
a control unit configured to control the blood pump and connected to the reader;
wherein the remote device comprises a user interface and a controller configured to perform a remote procedure comprising:
allowing entry of values for setting parameter values via the user interface, wherein the setting parameter values define a setting of the extracorporeal blood treatment apparatus and are related to an identified patient, the setting parameter values comprising part of a patient prescription for the identified patient, the setting parameter values comprising at least one of:
a patient fluid removal rate for the identified patient; and
two parameters for the identified patient selected from total weight loss, total treatment time, and ultrafiltration flow rate;
generating an optical pattern encoding the setting parameter values, the optical pattern being variable as a function of the entered setting parameter values;
wherein the disposable component comprises an identification code identifying the disposable component;
wherein the control unit of the extracorporeal blood treatment apparatus is configured to perform an acquisition procedure comprising:
acquiring the setting parameter values by reading the optical pattern through the reader;
setting the extracorporeal blood treatment apparatus based on the values of the setting parameter values.
the control unit of the extracorporeal blood treatment apparatus being further configured to perform a consistency procedure before setting the extracorporeal blood treatment apparatus based on the values of the setting parameter values, the consistency procedure comprising:
receiving the identification code of the disposable component to identify the disposable component, the control unit configured to receive the identification code from:
the reader of the extracorporeal blood treatment apparatus, wherein the identification code of the disposable component comprises a respective optical pattern; or
a frequency scanner, distinct from the reader of the apparatus, wherein the identification code of the disposable component comprises a frequency related signal;
verifying consistency between the identified disposable component and the setting parameter values acquired through reading the optical image from the display;
when one or more setting parameters are not consistent with the identified disposable component for the extracorporeal blood treatment apparatus, preventing the extracorporeal blood treatment apparatus to be set based on the setting parameter values.
